(19) United States
(12) Patent Application Publication
(10) Pub. No.: US 2011/0275576 A1
(43) Pub. Date: Nov. 10, 2011

(54) Carbohydrate-Cyclopamine Conjugates as Anticancer Agents

(75) Inventors: Cheng-Wei T Chang, Logan, UT (US); Massoud Garrossian, Plano, TX (US)

(73) Assignee: Utah State University, North Logan, UT (US)

(21) Appl. No.: 13/143,066

(22) PCT Filed: Jan. 6, 2010
(86) PCT No.: PCT/US10/20240

§ 371 Date: Jun. 30, 2011

Related U.S. Application Data
(60) Provisional application No. 61/142,821, filed on Jan. 6, 2009.

(51) Int. Cl. A61K 31/7052; A61P 35/00; C07H 5/04

(52) U.S. Cl. 514/23; 536/55

(57) ABSTRACT

Various anticancer compounds are disclosed, including a series of carbohydrate-cyclopamine conjugates. These compounds include pyranose, furanose, and mannitol conjugates, linked through a 1,2,3-triazine ring to the nitrogen of cyclopamine. Methods for preparing these compounds, pharmaceutical compositions containing these compounds, and their use as anticancer agents are also disclosed.

### TECHNICAL FIELD

The present disclosure relates the discovery and synthesis of cyclopamine derivatives linked via a triazine ring, to pharmaceutical compositions containing them, and to their use in the treatment of cancer.

### BACKGROUND OF THE INVENTION

Cyclopamine is an inhibitor of the Hedgehog signaling pathway, which directs the development of multiple tissues during embryonic development and which contributes to tissue homeostasis (i.e. cell growth and apoptosis) in adults. Excessive signaling in the Hedgehog pathway is associated with various types of human cancers, including those common to Gorlin syndrome (or nevoid basal cell carcinoma syndrome), such as medullablastomas, ovarian fibromas, and sarcomas. In addition, mutations in the receptor proteins in the Hedgehog pathway called “patched” and “smoothened”, have been linked to basal cell carcinoma, prostate cancer, small cell lung carcinoma, cancers of the upper gastrointestinal tract (including those of the esophagus, stomach, pancreas and biliary tract), and colon cancer. Cyclopamine has been shown to kill brain tumor cells in animals, as well. Regulation of cell growth or apoptosis may also be useful for the treatment of cancer or tumors of the head, neck, nasal cavity, paranasal sinuses, nasopharynx, oral cavity, oropharynx, larynx, hypopharynx, salivary glands, paragangliomas, liver, bone, intestine, rectum, breast, lymphatic system, blood, bone marrow, and central nervous system.

Cyclopamine itself has limited solubility in water, ethanol, methanol and DMSO, which may hinder its utility as a drug. In addition, to date, very little effort has been devoted into the synthesis of cyclopamine derivatives.

### SUMMARY OF THE INVENTION

We have discovered that compounds of Formula I:

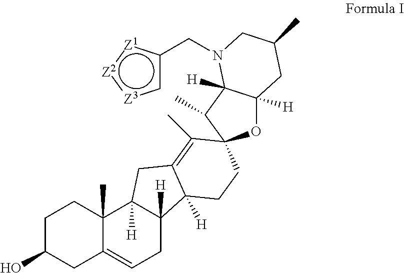

wherein:
Z¹-Z²-Z³ is N(R¹)-N═N or N═N-N(R¹); and
R¹ is at least one heterocycle optionally substituted with up to 7 substituents selected from the group consisting of H, OH, OR², SH, SR²,N(R²)₂, alkyl, and halogen, or
R¹ is at least one straight-chain saccharide optionally substituted with up to 7 substituents selected from the group consisting of H, OH, OR², SH, SR², N(R²)₂, alkyl, and halogen; and
R² is independently hydrogen, alkyl, aryl, acyl, aralkyl, or cycloalkyl; and pharmaceutically acceptable salts, hydrates, tautomers, dimers, solvates and complexes thereof,
   may be useful in the treatment ,of cancers. In some embodiments, these compounds may be useful in the treatment of cancers in the pancreas, esophagus, stomach, biliary tract, prostate, skin, lung, colon, and brain.

Certain embodiments of the compounds described by Formula I include those wherein R¹ is a saccharide or an oligosaccharide, and wherein R¹ is a pyranose. Other embodiments include compounds wherein R¹ is an α-pyranose, wherein R¹ is a furanose, and wherein R¹ is ribose. Further embodiments include compounds wherein R¹ is a straight-chain saccharide, and wherein R¹ is mannitol. Still other embodiments include dimers of the compounds of Formula I, where two cyclopamine cores are attached to a saccharide via two separate triazine ring linkages.

In one aspect of the disclosure, compositions are provided containing the present compounds in amounts for pharmaceutical use to treat mammalian cancer; such compositions may include a compound of Formula I in association with one or more pharmaceutically acceptable diluents, excipients and/or inert carriers. Such compounds or preparations may be administered systemically or locally. Some exemplary compositions of the invention exhibit increased solubility over cyclopamine.

### BRIEF DESCRIPTION OF THE DRAWINGS

FIG. 1 shows exemplary azido-heterocycles used in the cycloaddition step to synthesize compounds of Formula I.

FIG. 2 shows an exemplary method of synthesizing compounds of Formula I.

FIGS. 3(a)-3(f) show cells treated with an exemplary compound of Formula I disclosed in Example 3f, below, in comparison with cells treated with cyclopamine. A549 cells were incubated for 48 hours in the presence of (a) the compound of Example 3f, (hereafter “3f”) at 10 μM; (b) 3f at 30 μM; (c) 31 at 100 μM; (d) cyclopamine at 10 μM; (e) cyclopamine at 30 μM; (f) cyclopamine at 100 μM. Scale bar: 100 μm.

FIGS. 4-10 show the anticancer activity of the compounds of Examples 3a (FIG. 4), 3d (FIG. 5), 3e (FIG. 6), 3f (FIG. 7), 3h (FIG. 8), 3i (FIGS. 9), and 3k (FIG. 10), respectively, in a 60-cell line panel. A negative value for the growth percentage is an indication of anticancer activity.

### DETAILED DESCRIPTION OF THE INVENTION

This application claims priority to U.S. Provisional Pat. App. No. 61/142,821, filed 6 Jan. 2009, which is incorporated in its entirety by this reference.

Unless specified otherwise within this specification, the nomenclature used in this specification generally follows the examples and rules stated in A Guide to IUPAC Nomenclature of Organic Compounds (Recommendations 1993), 1993, Blackwell Scientific publications, which is incorporated by reference herein in its entirety for its exemplary chemical structure names and rules on naming chemical structures. Abbreviations generally follow those utilized by organic chemists of ordinary skill in the art, and a comprehensive list of these abbreviations are listed in the first issue each year of The Journal of Organic Chemistry, in a table entitled Standard Abbreviations and Acronyms, which is incorporated by reference herein in its entirety for its exemplary chemical abbreviations.

As used herein, the term “alkyl” refers to a saturated hydrocarbon group which includes straight-chained, branched, cyclic, alkyl-substituted cyclic and cycloalkyl-substituted alkyl groups. Exemplary alkyl groups include methyl (Me), ethyl (Et), propyl (including n-propyl, isopropyl, and cyclopropyl), butyl (including n-butyl, isobutyl, t-butyl, and cyclobutyl), and pentyl (including n-pentyl, isopentyl, and neopentyl) groups. In various embodiments, an alkyl group may contain from 1 to 20, from 1 to 10, from 1 to 8, from 1 to 6, from 1 to 4, or from 1 to 3 carbon atoms.

As used herein, “aralkyl” refers to an alkyl group substituted with an aryl group. Exemplary aralkyl groups include benzyl and phenethyl.

As used herein, “alkoxy” refers to an -O-alkyl or O-aralkyl group. Exemplarly alkoxy groups include methoxy, ethoxy, propoxy (e.g., n-propoxy and isopropoxy), and t-butoxy. Two alkoxy groups may be linked together through the alkyl component, such as with alkylenedioxy, isopropylidine, benzylidene or cyclohexylidene groups.

As used herein, “aryl” refers to an aromatic carbocycle group including monocyclic or polycyclic (e.g., having 2, 3 or 4 fused rings) aromatic hydrocarbons such as, for example, phenyl, naphthyl, anthracenyl, phenanthrenyl, indanyl, and indenyl. In some embodiments, aryl groups have from 6 to 20 carbon atoms.

As used herein, “aryloxy” refers to an -O-aryl group. Exemplary aryloxy groups are phenoxy and benzyloxy.

As used herein, “acyl” refers to a carboxylic acid group with the hydroxyl removed, to provide a substituted R(C═O) group. Exemplary acyl groups include formyl, acetyl, benzoyl and acetamido groups (linked through the amine).

As used herein, “alkynyl” refers to an alkyl group having one or more triple carbon-carbon bonds. Exemplary alkynyl groups include ethynyl and propynyl.

As used herein, “cycloalkyl” refers to an optionally substituted cyclic alkyl group and includes monocyclic and multiple ring structures such as bicyclic and tricyclic. In one embodiment, cycloalkyl has 3 to 6 carbon atoms. In another embodiment, the cycloalkyl is unsubstituted. Exemplary cycloalkyl groups include cyclopropyl and cyclobutyl.

As used herein, “halo” or “halogen” includes fluoro, chloro, bromo, and iodo, and includes both radioactive and non-radioactive forms.

As used herein, “heterocycle” refers to a saturated or unsaturated carbocycle group wherein one or more of the ring-forming carbon atoms of the carbocycle group is replaced by a heteroatom such as O, S, or N. Heterocycle groups can be aromatic (e.g., “heteroaryl”) or non-aromatic (e.g., “heterocycloalkyl”). Heterocycle groups can also correspond to hydrogenated and partially hydrogenated heteroaryl groups. Heterocycle groups can be characterized as having 3-18 ring-forming atoms. In certain embodiments, heterocycle groups can contain, in addition to at least one heteroatom, from 1 to 24, 2 to 10, or 2 to 5 carbon atoms and can be attached through a carbon atom or heteroatom. In further embodiments, the heteroatom can be oxidized (e.g., an S or N may have an oxo substituent) or a nitrogen atom can be quaternized. Exemplary heterocycle groups include carbohydrate or saccharide compounds, both in their furanose and pyranose forms, and oligosaccharides linked through 1Δ4 or 1→6 linkages. Also included are fused ring and spiro compounds containing, for example, the above heterocycles.

As used herein, the term “saccharide” includes straight-chain and/or cyclic carbohydrate groups containing 4, 5, 6 or 7 carbon atoms in their backbone, or polyhydroxy aldehydes or ketones. This term specifically includes the cyclic saccharide furanoses, pyranoses, and the straight-chain saccharide mannitol. It is understood that all cyclic and straight-chain forms of the carbohydrate carbon backbone, and all isomers (axial and equatorial) of each hydroxyl substituent are included unless noted otherwise, and includes both the α and β anomers. This also includes the (D) and (L) isomers of each saccharide or oligosaccharide.

The term “oligomer” refers to a short chain of saccharide units joined together by covalent bonds, containing from 2 to 50 monosaccharides. The oligomers may be linear or branched.

The term “optionally substituted” includes “not substituted” or “substituted with the same or different substituents”.

The term “protecting group” as used herein means temporary substituents which protect a potentially reactive functional group from undesired chemical reactions. Examples of such protecting groups include acetals and ketals, ethers, and esters of carboxylic acids. The field of protecting' group chemistry has been reviewed and is known to skilled artisans (T. W. Greene and P. Wuts, Protective Groups in Organic Synthesis, 2nd Ed.; Wiley; New York, 1991, which is incorporated herein by reference in its entirety).

Herein, the term “solvate” includes, for example, a solvate with an organic solvent, or a hydrate. A solvate with water is called a hydrate. When a solvate or hydrate is formed, any number of solvent or water molecules may be coordinated.

At various places in the present specification, substituents of compounds of some embodiments of the invention are disclosed in groups or in ranges. It is specifically intended that the invention include each and every individual subcombination of the members of such groups and ranges. For example, the term “C1-C6 alkyl” is specifically intended to individually disclose methyl, ethyl, propyl, butyl, pentyl, and hexyl. Also as an example, the term “C1-C4 alkyl” is specifically intended to individually disclose alkyl groups of C1-C2, C1-C3, C1-C4, C2-C3, C2-C4, and C3-C4.

For compounds of the invention in which a variable appears more than once, each variable can be a different moiety selected from the Markush group defining the variable. For example, where a structure is described having two R² groups that are simultaneously present on the same compound; the two R² groups can represent the same or different moieties selected from the Markush group defined for R².

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Compounds encompassed by the present invention include the compounds of Formula I:

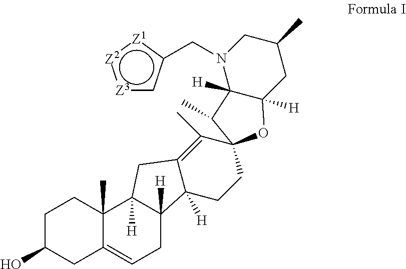

wherein:
Z¹-Z²-Z¹ is N(R¹)-N═N or N═N-N(R¹); and
R¹ is at least one heterocycle optionally substituted with up to 7 substituents selected from the group consisting of H, OH, OR², SH, SR², N(R²), and halogen, or
   R¹ is at least one straight-chain saccharide optionally substituted with up to 7 substituents selected from the group consisting of H, OH, OR², SH, SR², N(R²)₂, and halogen; and
      R² is independently hydrogen, alkyl, aryl, acyl, aralkyl, or cycloalkyl; and pharmaceutically acceptable salts, hydrates, tautomers, dimers, solvates and complexes thereof.

Certain embodiments of compounds of Formula I include those wherein R¹ is a saccharide. There may be multiple saccharide units (or oligosaccharides) contained in R¹, including disaccharides and trisaccharides, linked through 1→4 or 1→6 linkages, in various other embodiments. Further embodiments include those wherein R¹ is a pyranose. Other embodiments include those wherein R¹ is an α-pyranose. In certain embodiments, R¹ is an α-rhamnose. Further embodiments include those wherein R¹ is a furanose, and wherein R¹ is ribose. Other embodiments include those wherein R¹ is mannitol, and wherein R¹ is mannitol 1,6-linked to two cyclopamine cores through two independent triazine rings. In other embodiments, R¹ may also be a triose or tetrose.

Exemplary pyranoses include glucopyranose, mannopyranose, rhamopyranose, fucopyranose, xylopyranose, allopyranose, altropyranose, gulopyranose, idopyranose, galactopyranose, and talopyranose. Exemplary hexopentoses (straight-chain forms of the pyranoses) include allose, altrose, glucose, mannose, gulose, inose, galactose and talose.

Exemplary furanoses include ribofuranose, arabinofuranose, xylofuranose, lyxofuranose, and fructofuranose. Exemplary aldopentoses (straight-chain forms of the furanoses) include ribose, arabinose, xylose and lyxose.

Exemplary tetroses include erythrose and threose.

Exemplary trioses include glyceraldehyde.

Exemplary heptoses include sedoheptulose.

The heterocycle or heterocycles of R¹ of Formula I may be optionally substituted with various substituents. In certain embodiments, these substituents are saccharide protecting groups, including O-acetyl, N-acetyl, O-benzoyl, O-benzyl, O-methyl, O,O-diisopropylidine, O,O-dicyclohexylidene, and methyl-O-benzylidine. In further embodiments, these substituents are saccharide activating groups, including halogen, thiomethyl, thiophenyl, or inflate. Oligosaccharide synthesis or functionalization may occur before or after cycloaddition to the cyclopamine core, in various embodiments. After deprotection or hydrolysis of the saccharide protecting or activating groups, the substituents may be OH or NH₂, which may aid in rendering the compounds soluble in alcohol or aqueous media. In further embodiments, the saccharide protecting or activating groups themselves may aid solubility. In other embodiments, the saccharide may not contain protecting groups.

The compounds of Formula I may be synthesized by the following steps: addition of an alkyne component to the cyclopamine core; then reaction with an alkyl azide, in a 1,3-cycloaddition reaction, to form a cyclopamine conjugate linked through a triazine ring. The compounds resulting from the 1,3-cycloaddition may be regioisomeric, as the alkyl azide can add to the alkyne in two modes. Depending on the structure of the azido-compound coupled to the alkyne, production of one of the regioisomers may be favored. If necessary, the saccharide or heterocycle can be deprotected or further functionalized after the 1,3-cycloaddition step is completed.

In certain embodiments, a compound of Formula I, or a pharmaceutically acceptable salt, hydrate, or complex thereof, may be used in the manufacture of a medicament for the treatment of humans and other mammals. In further embodiments, the medicament may be for the treatment of cancer.

Compounds encompassed by the present invention also include the compounds of Formula II;

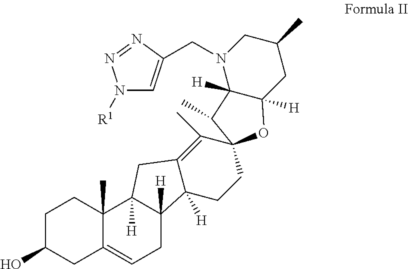

wherein:
R¹ is at least one heterocycle optionally substituted with up to 7 substituents selected from the group consisting of H, OH, OR², SH, SR², N(R²)₂, and halogen, or
R¹ is at least one straight-chain saccharide optionally substituted with up to 7 substituents selected from the group consisting of H, OH, OR², SH, SR², N(R²)₂, and halogen; and
   R² is independently hydrogen, alkyl, aryl, acyl, aralkyl, or cycloalkyl; and pharmaceutically acceptable salts, hydrates, tautomers, solvates and complexes thereof.

Certain embodiments of compounds of Formula II include those wherein R¹ is a saccharide. There may be multiple saccharide units (or oligosaccharides) contained in R¹, including disaccharides and trisaccharides, linked through 1→4 or 1→6 linkages, in various other embodiments. Further embodiments include those wherein R¹ is a pyranose. Other embodiments include those wherein R¹ is an α-pyranose. In certain embodiments, R¹ is an α-rhamnose. Further embodiments include those wherein R¹ is a furanose, and wherein R¹ is ribose. Other embodiments include those wherein R¹ is mannitol, and wherein R¹ is mannitol 1,6-linked to two cyclopamine cores through two independent triazine rings. In other embodiments, R¹ may also be a triose or tetrose.

Exemplary pyranoses include glucopyranose, mannopyranose, rhamopyranose, fucopyranose, xylopyranose, allopyranose, altropyranose, gulopyranose, idopyranose, galactopyranose, and talopyranose. Exemplary hexopentoses (straight-chain forms of the pyranoses) include allose, altrose, glucose, mannose, gulose, isose, galactose and talose.

Exemplary furanoses include ribofuranose, arabinofuranose, xylofuranose, lyxofuranose, and fructofuranose. Exemplary aldopentoses (straight-chain forms of the furanoses) include ribose, arabinose, xylose and lyxose.

Exemplary tetroses include erythrose and threose.

Exemplary trioses include glyceraldehyde.

Exemplary heptoses include sedoheptulose.

The heterocycle or heterocycles of R¹ of Formula II may be optionally substituted with various substituents. In certain embodiments, these substituents are saccharide protecting groups, including O-acetyl, N-acetyl, O-benzoyl, O-benzyl, O-methyl, O,O-diisopropylidine, O,O-dicyclohexylidene, and methyl-O-benzylidine. In further embodiments, these substituents are saccharide activating groups, including halogen, thiomethyl, thiophenyl, or triflate. Oligosaccharide synthesis or functionalization may occur before or after cycloaddition to the cyclopamine core, in various embodiments. After deprotection or hydrolysis of the saccharide protecting or activating groups, the substituents may be OH or NH₂, which may aid in rendering the compounds soluble in alcohol or aqueous media. In further embodiments, the saccharide protecting or activating groups themselves may aid solubility. In other embodiments, the saccharide may not contain protecting groups.

The compounds of Formula II may be synthesized by the following steps: addition of an alkyne component to the cyclopamine core, then reaction with an alkyl azide, in a 1,3-cycloaddition reaction, to form a cyclopamine conjugate linked through a triazine ring. If necessary, the saccharide or heterocycle can be deprotected or further functionalized after the 1,3-cycloaddition step is completed.

In certain embodiments, a compound of Formula II, or a pharmaceutically acceptable salt, hydrate, or complex thereof, may be used in the manufacture of a medicament for the treatment of humans and other mammals. In further embodiments, the medicament may be for the treatment of cancer.

Exemplary embodiments include:
N-(1-(2,3,4,6-tetra-O-acetyl-β-D-glucopyranosyl)-1H-1,2,3-triazol-4-yl)methylcyclopamine;
N-(1-(2,3,4,6-tetra-O-acetyl-(β-D-galactopyranosyl)-1H-1,2,3-triazol-4-yl)methylcyclopamine;
N-(1-(2,3,4,6-tetra-O-acetyl-α-D-mannopyranosyl)-1H-1,2,3-triazol-4-yl)methylcyclopamine;
N-(1-(4-O-(2,3,4,6-tetra-O-acetyl-β-D-galactopyranosyl)-2,3,6-tri-O-acetyl-β-D- glucopyranosyl)-1H-1,2,3-triazol-4-yl)methylcyclopamine;
N-(1-(2,3,4-tri-O-acetyl-β-L-fucopyranosyl)-1H-1,2,3-triazol-4-yl)methylcyclopamine;
N-(1-(2,3,4-tri-O-acetyl-α-L-rhamnopyranosyl)-1H-1,2,3-triazol-4-yl)methylcyclopamine;
N-(1-(2,3,4-tri-O-acetyl-α-D-xylopyranosyl)-1H-1,2,3-triazol-4-yl)methylcyclopamine;
N-(1-(2-acetamido-3,4,6-O-acetyl-2-deoxy-α-D-glucopyranosyl)-1H-1,2,3- triazol-4-yl)methylcyclopamine;
N-(1-(2-acetamido-3,4,6-tri-O-acetyl-2-deoxy-α-D-galactopyranosyl)-1H-1,2,3- triazol-4-yl)methylcyclopamine;
N-(1-(4-O-(2,3,4,6-tetra-O-acetyl-β-D-glucopyranosyl)-2,3,6-tri-O-acetyl-β-D- glucopyranosyl)-1H-1,2,3-triazol-4-yl)methylcyclopamine;
N-(1-(4-O-(4-O-(2,3,4,6-tetra-O-acetyl-β-D-glucopyranosyl)-2,3,6-tri-O-acetyl- β-D-glucopyranosyl)-2,3,6-tri-O-acetyl-β-D-glucopyranosyl))-1H-1,2,3-triazol-4-yl)methylcyclopamine;
N-(1-(β-D-glucopyranosyl)-1H-1,2,3-triazol-4-yl)methylcyclopamine;
N-(1-(D-galactopyranosyl)-1H-1,2,3-triazol-4-yl)methylcyclopamine;
N-(1-(α-D-mannopyranosyl)-1H-1,2,3-triazol-4-yl)methylcyclopamine;
N-(1-(4-O-(β-D-galactopyranosyl-β-D-glucopyranosyl)-1H-1,2,3-triazol-4-yl)methylcyclopamine;
N-(1-(β-L-fucopyranosyl)-1H-1,2,3-triazol-4-yl)methylcyclopamine;
N-(1-(α-L-rhamnopyranosyl)-1H-1,2,3-triazol-4-yl)methylcyclopamine;
N-(1-(α-D-xylopyranosyl)-1H-1,2,3-triazol-4-yl)methylcyclopamine;
N-(1-(2-acetamido-2-deoxy-α-D-glucopyranosyl)-1H-1,2,3-triazol -4-yl)methylcyclopamine;
N-(1-(2-acetamido-2-deoxy-α-D-galactopyranosyl)-1H-1,2,3-triazol-4-yl)methylcyclopamine;
N-(1-(4-O-(β-D-glucopyranosyl)-β-D-glucopyranosyl)-1H-1,2,3-triazol-4-yl)methylcyclopamime;
N-(1-(4-O-(4-O-(β-D-glucopyranosyl)-β-D-glucopyranosyl)-β-D- glucopyranosyl))-1H-1,2,3-triazol-4-yl)methylcyclopamine;
N-(1-(ribofuranosyl)-1H-1,2,3-triazol-4-yl)methylcyclopamine;
N-(1-(mannitol)-1H-1,2,3-triazol-4-yl)methylcyclopamine dimer; and
N-(1-(mannitol)-1H-1,2,3-triazol-4-yl)methylcyclopamine.

The novel compounds encompassed by the present invention can be prepared in a variety of ways known to one skilled in the art of organic synthesis. In some embodiments, the compounds of the present invention can be synthesized using the methods as hereinafter described below, together with synthetic methods known in the art of synthetic organic chemistry or variations thereon as appreciated by those skilled in the art.

One general synthetic scheme for the compounds of the present invention is as follows:

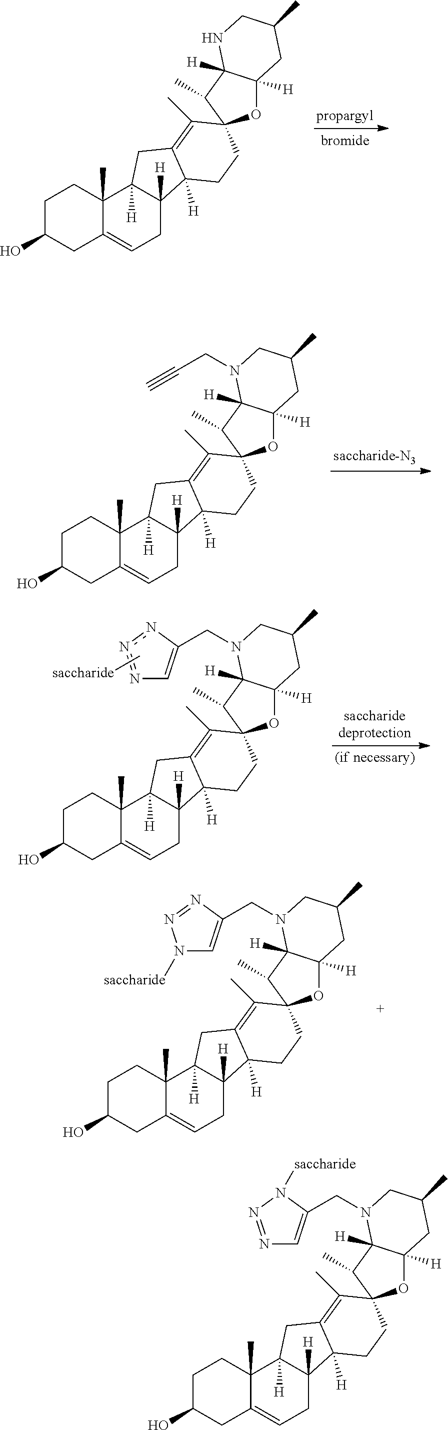

In general, compounds of Formula I or Formula II may be synthesized in two steps. The first step is addition of an alkyne component to the nitrogen atom of the cyclopamine core, which can be done with propargyl bromide in the presence of sodium carbonate. The functionalized cyclopamine core may then be reacted with an alkyl azide, in a 1,3-cycloaddition reaction, to form a cyclopamine conjugate. The second step can be done with a saccharide-azide in the presence of Cu(OAc)₂ and sodium ascorbate, using sonication. The compounds resulting from the 1,3-cycloaddition may be of two regioisomers, or configurations, in the triazine rimy, (shown above), as the azide can add to the alkyne in two modes. Depending on the structure of the saccharide, or other azido-compounds coupled to the alkyne, production of one of the conformers may be favored. In certain cases, only one regioisomer may be produced. If necessary, the saccharide or heterocycle can be deprotected or further functionalized after the 1,3-cycloaddition step is completed.

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatograpy (HPLC) or thin layer chromatography (TLC).

The reactions of the processes described herein can be carried out in suitable solvents which can be readily selected by one of skill in the art of organic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

A pharmaceutically acceptable salt of a compound of Formula I or Formula II includes a salt prepared from a pharmaceutically acceptable non-toxic base, such as an inorganic or organic base. A salt derived from an inorganic base is, for example, an aluminium, calcium, potassium, magnesium, sodium or zinc salt. A salt derived from an organic base is, for example, a salt of a primary, secondary or tertiary amine, such as arginine, betaine, benzathine, caffeine, choline, chloroprocaine, cycloprocaine, N′,N′-dibenzylethylenediamine, diethanolamine, diethylamine, 2-diethyl-aminoethanol, 2-dimethylaminoethanol, ethanolamine, ethylendiamine, N-ethyl-morpholine, N-ethyl piperidine, glutamine, glucosamine, histidine, hydrabamine, isopropylamine, lysine, meglumine, morpholine, piperazine, piperidine, polyamine resins, procaine, purines, tertiary butylamine, theobromine, triethylamine, trimethylamine, tripropylamine, tromethamine or thanolamine.

A pharmaceutically acceptable salt of a compound of Formula I or Formula II also includes a quaternary ammonium salt, for example where an amine group in a compound of Formula I reacts with a C1-C10 alkyl halide (for example, an alkyl chloride, bromide or iodide) to form a quaternary ammonium salt.

A pharmaceutically acceptable salt also includes a salt of pharmaceutically acceptable organic acid, such as a carboxylic or sulphonic acid, for example: an acetate, adipate, alginate, ascorbate, aspartate, benzenesulphonate (besylate), benzoate, butyrate, camphorate, camphorsulphonate (such as [(1S,4R)-7,7-dimethyl-2-oxobicyclo[2.2.1]hept-1-yl]methanesulfonic acid salt), camsylate, citrate, p-chlorobenzenesulphonate, cyclopentate, 2,5-dichlorobesyalte, digluconate, edisylate (ethane-1,2-disulfonate or ethane-1-(sulfonic acid)-2-sulfonate), esylate, ethanesulphonate, fumarate, formate, 2-furoate, 3-furoate, gluconate, glucoheptanate, glutamate, glutarate, glycerophosphate, glycolate, heptanoate, hexanoate, hippurate, 2-hydroxyethane sulfonate, lactate, lactobionate, laurate, malate, maleate, malonate, mandelate, methanesulphonate, 2-naphthalenesulfonate, napadisylate (naphthalene-1,5-disulfonate or naphthalene-1-(sulfonic acid)-5-sulfonate), nicotinate, oleate, orotate, oxalate, pantothenate, pamoate, pamoic, pectinate, 3-phenylpropionate, pivalate, propionate, pivolate, pyruvate, saccharinate, salicylate, stearate, succinate, tartrate, p-toluenesulphonate, transcinnamic acid, trifluoroacetate, xinafoate, xinofolate, xylate (p-xylene-2-sulphonic acid), undecanoate, 2-mesitylenesulphonate, 2-naphthalenesulphonate, D-mandelate, L-mandelate, 2,5-dichlorobenzenesulphonate, cinnamate or benzoate; or a salt of an inorganic acid such as a hydrobromide, hydrochloride, hydroiodide, sulphate, bisulfate, phosphate, nitrate, hemisulfate, thiocyanate, persulfate, phosphate or sulphonate salt. In another aspect of the invention the stoichiometry of the salt is, for example, a hemi-salt, or a mono- or di-salt or tri-salt.

A pharmaceutically acceptable salt, of a compound of Formula I or Formula II can be prepared in situ during the final isolation and purification of a compound, or by separately reacting the compound with a suitable organic or inorganic acid and isolating the salt thus formed.

The neutral forms of the compounds of certain embodiments of the present invention may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

Other embodiments of the present invention possess chiral or asymmetric carbon atoms (optical centers), double bonds, and anomeric centers; the racemates, diastereomers, geometric isomers, anomers and individual optical isomers are all intended to be encompassed within the scope of the present disclosure.

Certain embodiments of the invention may also include all isotopes of atoms occurring in the intermediates or final compounds. Isotopes include those atoms having the same atomic number but different mass numbers. For example, isotopes of hydrogen include tritium and deuterium.

Certain embodiments of the invention may also include tautomeric forms, such as keto-enol tautomers. Tautomeric forms can be in equilibrium or sterically locked into one form by appropriate substitution. Certain embodiments containing saccharide heterocycles may also include conformational isomers, such as boat or chair conformations, as well as a and β isomers at the anomeric position of the saccharide.

Some of the compounds of the invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

In addition to salt forms, the present disclosure provides compounds which may be in a prodrug form. Prodrugs of the compounds described herein are those compounds that readily undergo chemical changes under physiological conditions to provide the compounds of the present invention. Additionally, prodrugs can be converted to the compounds of the present disclosure by chemical or biochemical methods in an ex-vivo environment. For example, prodrugs can be slowly converted to the compounds of the present invention when placed in a transdermal patch reservoir with a suitable enzyme or chemical reagent. In certain embodiments, a prodrug moiety may be attached to the C-3 hydroxy group of the cyclopamine core. In other embodiments, a prodrug moiety may be attached to the heterocycle portion of the conjugate. In still other embodiments, a prodrug moiety may be attached to the saccaride, for example, as a substituent on the pyranose ring.

In order to use a compound of Formula I or Formula II, or a pharmaceutically acceptable salt or complex thereof, for the treatment of humans and other mammals, it is normally formulated in accordance with standard pharmaceutical practice as a pharmaceutical composition.

The compounds of Formula I or Formula II may be administered alone, or may be combined with a pharmaceutically-acceptable diluent and/or carrier or excipient according to standard pharmaceutical practice. For the oral mode of administration, the compounds disclosed are used in the form of tablets, capsules, lozenges, chewing gum, troches, powders, syrups, elixirs, aqueous solutions and suspensions, and the like. In the case of tablets, carriers that are used include lactose, sodium citrate and salts of phosphoric acid. Various disintegrants such as starch, and lubricating agents such as magnesium stearate and talc, are commonly used in tablets. For oral administration in capsule form, useful diluents are lactose and high molecular weight polyethylene glycols. If desired, certain sweetening and/or flavoring agents are added. For parenteral administration, sterile solutions of the compounds of the invention are usually prepared, and the pHs of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzylchromium chloride, and the usual quantities of diluents and/or carriers. For pulmonary administration, diluents and/or carriers will be selected to be appropriate to allow the formation of an aerosol.

The anticancer compounds of the invention may be administered by different routes including intravenous, intraperitoneal, subcutaneous, intramuscular, oral, topical (transdermal), or transmucosal administration. For systemic administration, oral administration may be used.

Alternatively, injection (parenteral administration) may be used, e.g., intramuscular, intravenous, intraperitoneal, or subcutaneous. For injection, the anticancer compounds are formulated in liquid solutions, such as in physiologically compatible buffers or solutions, such as saline solution, Hank's solution, or Ringer's solution. In addition, the compounds may he formulated in solid form and redissolved or suspended immediately prior to use. Lyophilized forms can also be produced.

Systemic administration can also be achieved by transmucosal or transdermal methods. For transmucosal or transdermal administration, penetrants appropriate to the barrier to be permeated may be used in the formulation. Such penetrants are generally known in the art, and include, for example, for transmucosal administration, bile salts and fusidic acid derivatives. In addition, detergents may be used to facilitate permeation. Transmucosal administration, for example, may be through nasal sprays, rectal suppositories, or vaginal suppositories.

For topical administration, the anticancer compounds can be formulated into ointments, salves, gels, or creams, as is generally known in the art.

Compositions of Formulae I or II and their pharmaceutically acceptable salts and/or complexes, which are active when given orally, can be formulated as syrups, tablets, capsules, and lozenges. A syrup formulation will generally consist of a suspension or solution of the compound or salt in a liquid carrier such as, for example, ethanol, peanut oil, olive oil, glycerine or water with a flavoring or coloring agent. Where the composition is in the form of a tablet, any pharmaceutical carrier routinely used for preparing solid formulations may be used. Examples of such carriers include magnesium stearate, terra alba, talc, gelatin, acacia, stearic acid, starch, lactose and sucrose. Where the composition is in the form of a capsule, any routine encapsulation is suitable, for example using the aforementioned carriers in a hard gelatin capsule shell. Where the composition is in the form of a soft gelatin shell capsule, any pharmaceutical carrier routinely used for preparing dispersions or suspensions may be utilized. For example, aqueous gums, celluloses, silicates or oils may be used to form a soft gelatin capsule shell.

Typical parenteral compositions consist of a solution or suspension of a compound or salt in a sterile aqueous or non-aqueous carrier optionally containing parenterally acceptable oil, for example polyethylene glycol, polyvinylpyrrolidone, lecithin, arachis oil or sesame oil.

Typical compositions for inhalation are in the form of a solution, suspension or emulsion that may be administered as a dry powder or in the form of an aerosol using a conventional propellant such as dichlorodifluoromethane or trichlorofluoromethane, or a non-fluorinated propellant.

A typical suppository formulation comprises a compound of Formula I or Formula II or a pharmaceutically acceptable salt or complex thereof which is active when administered in this way, with a binding and/or lubricating agent, for example polymeric glycols, gelatins, cocoa-butter or other low-melting vegetable waxes or fats or their synthetic analogs.

Typical dermal and transdermal formulations comprise a conventional aqueous or non-aqueous vehicle, for example a cream, ointment, lotion or paste or are in the form of a medicated plaster, patch or membrane.

The composition may be in unit dosage form, for example a tablet, capsule or metered aerosol dose, so that the patient may administer a single dose.

No unacceptable toxological effects are expected when the compounds disclosed are administered in accordance with the methods described.

Numerous administration vehicles will be apparent to those of ordinary skill in the art, including without limitation slow release formulations, liposomal formulations and polymeric matrices.

The amount of an anticancer compound of the invention to be administered can be determined by standard procedures taking into account factors such as the compound IC₅₀, the biological half-life of the compound, the age, size and weight of the patient, and the type of cancer associated with the patient. The significance of these and other factors to be considered are known to those of ordinary skill in the art and are used to determine an effective amount of the compound for each patient. In certain embodiments, an effective amount is determined by an IC₅₀ value of less than 200 uM in the MTS assay described herein. In other embodiments, an effective amount is determined by an IC₅₀ value of less than 100 uM in the MTS assay described herein. In further embodiments, an effective amount is determined by an IC₅₀ value of less than I uM in the MTS assay described herein.

Amounts administered also depend on the routes of administration and the degree of oral bioavailability. For example, for compounds with low oral bioavailability, relatively higher doses may have to be administered.

The composition may be in unit dosage form. For oral application, for example, a tablet or capsule may be administered, for nasal application, a metered aerosol dose may be administered, for transdermal application, a topical formulation or patch may be administered, and for transmucosal delivery, a buccal patch may be administered. In each case, dosing is such that the patient may administer a single dose.

Each dosage unit for oral administration may contain from 0.01 to 500 mg/Kg, such as from 0.1 to 50 mg/Kg, of a compound of Formulae I or II, or a pharmaceutically acceptable salt or complex thereof. The daily dosage for parenteral, nasal, oral inhalation, transmucosal or transdermal routes may contain from 0.01 mg to 100 mg/Kg, of a compound of Formula I or Formula II. A topical formulation may contain 0.01 to 5.0% of a compound of Formula I or Formula II. The active ingredient may be administered as a single dose or in multiple does, for example, from 2 to 6 times per day, sufficient to exhibit the desired activity, as is readily apparent to one skilled in the art.

As used herein, “treatment” of a disease includes, but is not limited to prevention, retardation and prophylaxis of the disease.

Diseases and disorders which may be treated or prevented, based upon regulation of cell growth or apoptosis, include Gorlin syndrome (or nevoid basal cell carcinoma syndrome), medullablastomas, ovarian fibroma, sarcoma, basal cell carcinoma, prostate cancer, small cell lung carcinoma, cancers of the upper gastrointestinal tract (including those of the esophagus, stomach, pancreas and biliary tract), colon cancer, skin cancer, brain cancer, and cancer or tumors of the head, neck, nasal cavity, paranasal sinuses, nasopharynx, oral cavity, oropharynx, larynx, hypopharynx, calivary glands, paragangliomas, liver, bone, intestine, rectum, breast, lymphatic system, blood, bone marrow, and central nervous system.

Another aspect of the present disclosure includes a method of treating a patient comprising administering to the patient an amount of a compound of Formula I or Formula II sufficient to treat humans and other mammals. In further embodiments, the method may be for the treatment of humans and other mammals that have cancer.

Other embodiments of the disclosure include a method of treating cancer that comprises the selection of compounds of Formula I or Formula II, extracting cancer cells from a host organism (a patient with cancer), evaluating the ability of the compounds of Formula I or Formula II to reduce the proliferation of the cells using the MTS assay, and when a compound is found that is effective at reducing the proliferation of the cells in the MTS assay, using an effective amount of that compound to treat the host.

### EXAMPLES

The Examples herein are to be construed as merely illustrative and not a limitation of the scope of this disclosure in any way, although the specifics recited herein may include independently patentable subject matter. The reagents and intermediates used in the following examples are either commercially available or can be prepared according to standard literature procedures by those skilled in the art of organic synthesis.

Proton magnetic resonance spectra were recorded using a Bruker 400 MHz spectrometer. Chemical shifts were reported as parts per million (ppm) downfield from tetramethylsilane in δ units, and J_{AB} coupling constants are reported in Hz. Splitting patterns were designed as s, singlet; d, doublet; dd, doublet of doublets; t, triplet; q, quartet; m, multiplet. ¹³C spectra were obtained using the Bruker 400 spectrometer at 100 MHz. Routine ¹³C NMR spectra were fully decoupled by broad-band waltz decoupling. All NMR spectra were recorded at ambient temperature unless otherwise noted.

Chemical reagents and starting materials were purchased from Aldrich Chemical Co. or Acros Chemical Co. and were used without purification unless otherwise noted. Dichloromethane was distilled over CaH₂. Other solvents were used without purification.

### Example 1

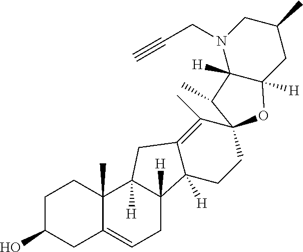

N-Propargylcyclopamine (Example 1). To a solution of cyclopamine (0.070 g, 0.17 mmol) and NaHCO₃ (0.021 g, 0.26 mmol) in MeCN (25 mL), propargyl bromide (0.028 mL, 0.26 mmol, 80% in toluene) was added. The reaction mixture was refluxed for 2.5 hrs. After completion of the reaction as determined by TLC (eluted with MeOH/EtOAc=3/7), the reaction mixture was cooled to room temperature and then stored in the freezer overnight. The solution was carefully removed by pipet. To the residue was added hexane (5 mL) and the solution was cooled in the freezer. After removal of the hexane by pipet, the residue was pump-dried and then recrystallized from DCM and hexane. The purified product was obtained as white solid (0.072 g, 0.16 mmol, 94%). ¹H NMR (CDCl₃, 400 MHz) δ 5.37 (m, 1H), 4.33 (m, 1H), 3.2-3.6 (m, 4H), 2.86 (dd, J=3.5, 11.1 Hz, 1H), 2.4 (m, 2H), 2.2 (m, 4H), 2.0 (m, 2H), 1.6-1.8 (m, 10H), 1.4-1.6 (m, 2H), 1.2-1.4 (m, 6H), 0.8-1.2 (m, 10H); ¹³C NMR (CDCl₃, 100 Hz) δ 143.1, 141.8, 127.0, 122.1, 84.9, 78.5, 74.1, 73.8, 72.0, 68.7, 61.3, 52.2, 49.5, 43.9, 42.0, 41.7, 39.5, 38.3, 38.2, 36.7, 33.1, 31.6, 31.3, 29.5, 29.2, 24.8, 19.3, 18.9, 13.7, 10.9; HRESI/APCI Calcd for C₃₀H₄₄NO₂ ([MH]⁺) m/e 450.3372; measured m/e 450.3374.

General procedure for the 1,3-dipolar cycloaddition of propargylcyclopamine and an alkyl azide for Examples 2a-2k. N-Propargylcyclopamine (0.020 g, 0.044 mmol), the alkyl azide (1.2 equiv.), Cu(OAc)₂ (2.0 equiv.), and sodium ascorbate (0.5 equiv.) were added to a vial containing MeOH (1 7 mL), THF (0.46 mL) and H₂O (0.3 mL). The reaction was sonicated for a total of 28 minutes (4×7 minutes). After completion of the reaction as determined by TLC (eluted with 65% EtOAc and 35% hexane), the solvents were removed and the crude product was purified by flash column chromatography (hexane/EtOAc=1/1 to EtOAc). The purified products were obtained as white solids.

### Example 2a

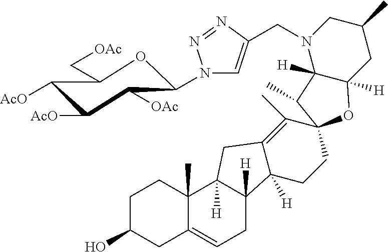

N-(1-(2,3,4,6-Tetra-O-acetyl-β-D-glucopyranosyl)-1H-1,2,3-triazol-4-yl)methylcyclopamine (2a). The general procedure for the 1,3-dipolar cycloaddition of propargylcyclopamine and an alkyl azide was used to prepare Example 2a. ¹H NMR (CDCl₃, 400 MHz) δ 7.74 (s, 1H), 5.87 (d, J=9.1 Hz, 1H), 5.4 (m, 4H), 5.2-5.3 (m, 1H), 4.32 (dd, J=12.7 Hz, J=5.2 Hz, 1H), 4.1 (m, 1H), 4.0 (m, 2H), 3.5-3.6 (m, 4H), 2.4 (s, 1H), 2.3 (s, 1H), 2.2 (m, 1H), 2.1 (m, 4H), 2.0-2.1 (m, 3H), 2.09 (s, 3H), 2.08 (a, 3H), 2.04 (s, 3H), 1.83 (s, 3H), 0.7-1.7 (m, 25H); ¹³C NMR (CDCl₃, 100 Hz) δ 170.7, 170.1, 169.6, 170.0, 143.2, 141.8, 127.1, 122.1, 121.2, 117.4, 86.0, 85.0, 75.4, 72.7, 72.0, 70.6, 68.0, 61.9, 52.2, 49.5, 42.0, 41.7, 39.8, 38.3, 38.2, 36.8, 33.1, 32.1, 32.0, 31.6, 31.3, 30.5, 29.9, 29.5, 29.2, 24.9, 20.9, 20.8, 20.7, 20.4, 19.2, 18.9, 13.7, 10.9; HRESI/APCI Calcd for C₄₄H₆₃N₄O₁₁ ([MH]⁺) m/e 823.4493; measured m/e 823.4515.

### Example 2b

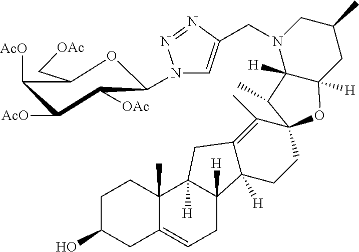

N-(1-(2,3,4,6-Tetra-O-acetyl-(β-D-galactopyranosyl)-1H-1,2,3-triazol-4-yl)methylcyclopamine (2b). The general procedure for the 1,3-dipolar cycloaddition of propargylcyclopamine and an alkyl azide was used to prepare Example 2b. ¹H NMR (CDCl₃,400 MHz) δ 7.91 (s, 1H), 5.81 (d, J=9.4 Hz, 1H), 5.5 (m, 2H), 5.4 (m, 1H), 5.23 (d, =10.0 Hz, 1H), 4.2 (m, 5H), 4.0 (s, 1H), 3.5 (m, 4H), 2.8 (m, 1H), 2.6 (m, 1H), 2.3 (m, 1H), 2.22 (s, 3H), 2.0-2.2 (m, 8H), 2.02 (s, 3H), 1.98 (s, 3H), 1.85 (s, 3H), 0.8-1.8 (m, 23H); ¹³C NMR (CDCl₃, 100 Hz) δ 170.5, 170.2, 170.0, 169.2, 143.2, 141.8, 128.9, 128.6, 127.9, 127.1, 122.1, 86.6, 85.0, 74.4, 72.3, 70.8, 68.1, 67.1, 61.4, 52.2, 49.5, 42.0, 41.7, 38.4, 36.8, 33.1, 32.1, 31.6, 31.3, 29.9 (2 carbons), 29.6, 29.2, 24.8, 22.9, 20.9, 20.9, 20.7, 20.5, 19.3, 18.9, 14.3, 13.7, 11.0; HRESI/APCI Calcd for C₄₄H₆₃N₄O₁₁ ([MH]⁺) m/e 823.4493; measured m/e 823.4503.

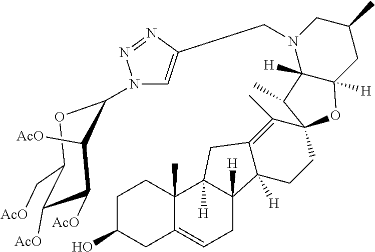

### Example 2c

N-(1-(2,3,4,6-Tetra-O-acetyl-α-D-mannopyranosyl)-1H-1,2,3-triazol-4-yl)methylcyclopamine (2c). The general procedure for the 1,3-dipolar cycloaddition of propargylcyclopamine and an alkyl azide was used to prepare Example 2c. ¹H NMR (CDCl₃, 400 MHz) δ 7.90 (s, 1H), 6.17 (s, 1H), 5.70 (s, 1H), 5.3 (m, 4H), 4.32 (dd, J=12.7 Hz, J=6.2 Hz, 1H), 4.20 (d, J=12.1 Hz, 1H), 4.10 (dd, J=14.2 Hz, J=7.0 Hz, 1H), 3.97 (m, 2H), 3.53 (m, 4H), 2.9 (m, 1H), 2.6 (m, 1H), 2.3 (m, 2H), 2.0-2.2 (m, 7H), 2.07 (s, 6H), 1.98 (s, 3H), 1.93 (s, 3H), 0.8-1.8 (m, 23H); ¹³C (CDCl₃, 100 Hz) δ 170.7, 169.9, 169.8, 169.1, 143.4, 141.8 (2 carbons), 126.8, 122.1 (2 carbons), 85.1, 76.1, 72.0, 70.9, 69.2, 65.2, 62.0, 60.6, 52.2, 49.5, 42.0, 41.7, 39.8, 38.3, 38.2, 36.7, 33.0, 31.6, 31.3, 30.2, 29.3, 24.8, 22.9, 21.3, 21.0, 20.9, 20.8, 20.7, 19.2, 18.9, 14.4, 13.7, 11.4; HRESI/APCI Calcd for C₄₄H₆₃N₄O₁₁ ([MH]⁺) m/e 823.4493; measured m/e 823.4497.

### Example 2d

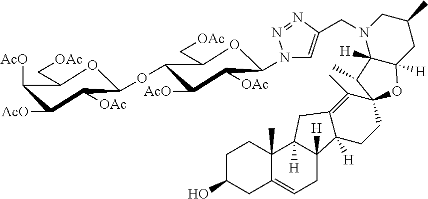

N-(1-(4-O-(2,3,4,6-Tetra-O-acetyl-β-D-galactopyranosyl)-2,3,6-tri-O-acetyl-β-D- glucopyranosyl)-1H-1,2,3-triazol-4-yl)methylcyclopamine (2d). The general procedure for the 1,3-dipolar cycloaddition of propargylcyclopamine and an alkyl azide was used to prepare Example 2d. NMR (CDCl₃, 400 MHz) δ 7.64 (s, 1H), 5.79 (d, J=8.9 Hz, 1H), 5.4 (m, 4H), 5.11 (dd, J=10.3 Hz, J=9.5 Hz, 1H), 4.96 (dd, J=10.4 Hz, J=3.4 Hz, 1H), 4.5 (m, 2H), 4.0-4.2 (m, 4H), 3.9-4.0 (m, 4H), 3.4-3.5 (m, 4H), 2.4 (s, 1H), 2.3 (s, 1H), 2.2 (m, 2H), 2.2 (m, 5H), 2.22 (3H), 2.20 (s, 3H), 2.14 (m, 3H), 2.06 (s, 6H), 2.01 (s, 1H), 1.95 (s, 3H), 1.83 (s, 3H), 0.7-1.7 (m, 25H); ¹³C NMR (CDCl₃, 100 Hz) δ 170.6, 170.4, 170.3 (2 carbons), 169.6, 169.3 (2 carbons), 143.2, 141.8, 133.5, 127.1, 122.1, 119.2, 101.3, 85.8, 85.0, 76.2, 75.9, 72.6, 72.0, 71.1, 69.2, 66.8, 62.0, 61.0, 52.2, 49.5, 42.0, 41.7, 39.8, 38.3, 38.2, 36.8, 33.1, 32.1, 32.0, 31.6, 31.3, 32.1, 31.6, 29.9 (2 carbons), 29.2, 24.8, 22.9, 21.0, 20.9 (2 carbons), 20.8 (2 carbons), 20.7, 20.4, 19.2, 18.9, 13.7, 10.9; HRESI/APCI Calcd for C₅₆H₇₉N₄O₁₉ ([MH]⁺) m/e 1111.5339; measured m/e 1111.5320.

### Example 2e

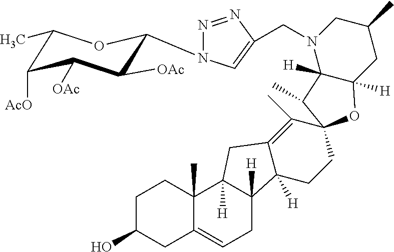

N-(1-(2,3,4-Tri-O-acetyl-β-L-fucopyranosyl)-1H-1,2,3-triazol-4-yl)methylcyclopamine (2e). The general procedure for the 1,3-dipolar cycloaddition of propargylcyclopamine and an alkyl azide was used to prepare Example 2e. ¹H NMR (CDCl₃, 400 MHz) δ 7.85 (s, 1H), 5.80 (d, J=9.2 Hz, 1H), 5.5 (m, 1H), 5.4 (m, 3H), 5.3 (m, 2H), 4.1 (m 2H), 4.0 (m, 1H), 3.9 (m, 1H), 4.4-4.6 (m, 5H), 2.80 (s, 1H), 2.6 (m, 2H), 2.2 (m, 2H), 2.25 (s, 3H), 2.01 (s, 3H), 1.88 (s, 3H), 0.7-1.9 (m, 29H); ¹³C NMR (CDCl₃, 100 Hz) δ 170.5, 170.0, 169.4, 143.0, 141.8 (2 carbons), 127.0, 122.1 (2 carbons), 86.7, 85.0, 73.0, 72.0, 71.3, 70.1, 68.4, 52.3, 49.6, 52.3, 49.6, 42.0, 41.7, 38.4, 36.8, 32.1, 31.6, 31.3, 29.9 (2 carbons), 29.5, 29.2, 24.8, 22.9, 20.9, 20.7, 20.5, 19.3, 18.9, 16.3, 14.3, 13.7, 11.0; HRESI/APCI Calcd for C₄₂H₆₁N₄O₉ m/e 765.4439; measured ink 765.4433.

### Example 2f

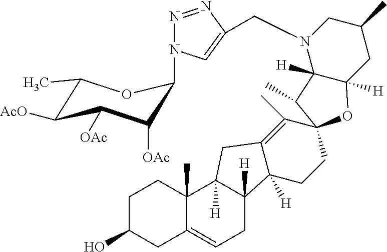

N-(1-(2,3,4-Tri-O-acetyl-α-L-rhamnopyranosyl)-1H-1,2,3-triazol-4-yl)methylcyclopamine (2f). The general procedure for the 1,3-dipolar cycloaddition of propargylcyclopamine and an alkyl azide was used to prepare Example 2f. ¹H NMR (CDCl₃, 400 MHz) δ 7.61 (s, 1H), 6.09 (s, 1H), 5.70 (s, 1H), 5.35 (s, 1H), 5.2 (m, 3H), 3.90 (s, 1H), 3.8 (m, 1H), 3.5 (m, 3H), 2.4 (m, 1H), 2.3 (m, 1H), 2.4 (m, 2H), 2.0-2.2 (m, 6H), 2.12 (s 3H), 2.07 (s, 3H), 1.96 (s, 3H), 0.7-1.9 (m, 29H); ¹³C NMR (CDCl₃, 100 Hz) δ 170.0 (2 carbons), 169.1, 143.3, 141.8 (2 carbons), 126.9, 122.1 (2 carbons), 85.0, 74.3, 74.1, 72.0, 71.0, 69.9, 69.5, 52.3, 49.5, 42,0, 41.7, 39.9, 38.4, 38.3, 36.8, 33.0, 32.1,31.6, 31.3, 29.9 (2 carbons), 29.2, 24.8, 22.9, 20.9, 20.7, 20.7, 19.2, 18.9, 17.7, 14.3, 13.7, 11.1; HRESI/APCI Calcd for C₄₂H₆₁N₄O₉ ([MH]⁺) m/e 765.4439; measured m/e 765.4422.

### Example 2g

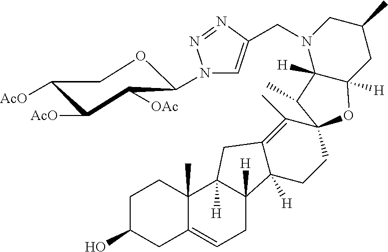

N-(1-(2,3,4-Tri-O-acetyl-α-D-xylopyranosyl)-1H-1,2,3-triazol-4-yl)methylcyclopamine (2g). The general procedure for the 1,3-dipolar cycloaddition of propargylcyclopamine and an alkyl azide was used to prepare Example 2g. ¹H NMR (CDCl₃, 400 MHz) δ 7.68 (s, 1H), 5.75 (d, J=9.0 Hz, 1H), 5.4 (m, 4H), 5.1 (m, 1H), 4.28 (dd, J=11.5 Hz, J=5.7 Hz, 1H), 4.1 (dd, J=14.4 Hz, J=7.2 Hz, 1H), 3.8 (m, 1H), 3.4-3.6 (m, 4H), 2.8 (m, 1H), 2.6 (m, 1H), 2.2-2.3 (m, 6H), 2.0-2.2 (m, 12H), 0.7-1.9 (m, 24H); ¹³C NMR (CDCl₃, 100 Hz) δ 170.1, 170.0, 169.1, 143.1, 141.8, 127.0, 122.1, 121.2, 117.3, 86.6, 85.0, 74.2, 72.2, 72.0, 70.8, 68.7, 65.9, 52.2, 49.5, 42.0, 41.7, 39.8, 38.3, 38.2, 36.8, 33.1, 31.6, 31.3, 29.9 (2 carbons), 29.6, 29.2, 24.8, 22.9, 20.8, 20.4, 19.2, 18.9, 14.3, 13.7, 10.9; HRESI/APCI Calcd for C₄₁ H₅₉N₄O₉([MH]⁺) m/e 751.4282; measured m/e 751.4276.

### Example 2h

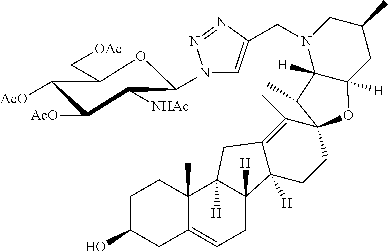

N-(1-(2-Acetamido-3,4,6-tri-O-acetyl-2-deoxy-α-D-glucopyranosyl)-1H-1,2,3- triazol-4-yl)methylcyclopamine (2h). The general procedure for the 1,3-dipolar cycloaddition of propargylcyclopamine and an alkyl azide was used to prepare Example 2h. ¹H NMR (CDCl₃, 400 MHz) δ 7.81 (s, 1H), 5.94 (d, J=9.8 Hz, 1H), 5.40 (m, 1H), 5.25 (t, J=9.8 Hz, 1H), 4.6 (m, 1H), 4.30 (dd, J=12.7 Hz, J=5.1 Hz, 1H), 4.2 (m, 2H), 4.0 (m, 3H), 3.5 (m, 4H), 2.9 (m, 1H), 2.7 (m, 1H), 2.3 (m, 1H), 2.1-2.2 (m, 4H), 2.10 (s, 3H), 2.06 (s, 3H), 2.05 (s, 3H), 1.9 (m, 2H), 1.8 (s, 3H), 0.7 - 1.7 (m, 27H); ¹³C NMR (CDCl₃, 100 Hz) δ 171.1, 170.7, 170.3, 169.5, 147.1, 141.8, 127.1, 122.1 (2 carbons), 117.4, 86.4, 85.0, 75.4, 74.2, 72.6, 72.0, 68.1, 62.0, 53.7, 52.3, 49.5, 42.0, 41.7, 39.8, 38.4, 38.2, 36.8, 33.1, 32.0, 31.8, 31.6, 31.3, 29.9, 29.5, 29.2, 24.8, 23.1, 20.9, 20.8 (2 carbons), 19.2, 18.9, 13.7, 10.9; HRESI/APCI Calcd for C₄₄H₆₄N₅O₁₀ ([MH]⁺) m/e 822.4653; measured m/e 822.4644.

### Example 2i

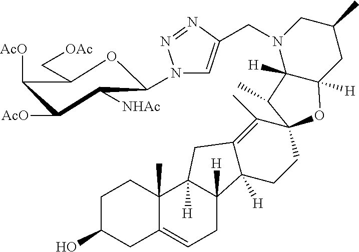

N-(1-(2-Acetamido-3,4,6-tri-O-acetyl-2-deoxy-α-D-galactopyranosyl)-1H-1,2,3- triazol-4-yl)methylcyclopamine (2i). The general procedure for the 1,3-dipolar cycloaddition of propargylcyclopamine and an alkyl azide was used to prepare Example 2i. ¹H NMR (CDCl₃, 400 MHz) δ 7.90 (s, 1H), 6.39 (m, 2H), 6.0 (d, J=9.6 Hz, 1H), 5.4 (m, 7H), 4.6 (m, 2H), 4.1 (m, 10H), 3.5 (m, 4H), 2.9 (m, 1H), 2.6 (m, 1H), 0.8-2.2 (m, 34H); ¹³C NMR (CDCl₃, 100 Hz) δ 171.0, 170.8, 170.4, 169.5, 143.2, 141.8 (2 carbons), 126.9, 122.1 (2 carbons), 95.8, 85.0, 78.7, 75.5, 75.2, 70.5, 68.2, 62.8, 62.4, 60.6, 58.0, 52.2, 49.5, 42.0, 41.7, 38.4, 36.7, 33.0, 31.5, 31.3, 29.9, 29.4, 29.2, 24.8, 23.3, 22.9, 21.3, 21.0, 20.9, 19.3, 18.9, 14.4, 13.7, 11.0; HRESI/APCI Calcd for C₄₄H₆₄N₅O₁₀ ([MH ]⁺) m/e 822.4653: measured m/e 822.4646.

### Example 2j

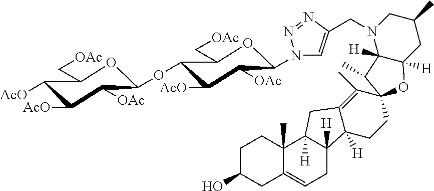

N-(1-(4-O-(2,3,4,6-Tetra-O-acetyl-β-D-glucopyranosyl)-2,3,6-tri-O-acetyl-β-D- glucopyranosyl)-1H-1,2,3-triazol-4-yl)methylcyclopamine (2j). The general procedure for the 1,3-dipolar cycloaddition of propargylcyclopamine and an alkyl azide was used to prepare Example 2j. NMR (CDCl₃, 400 MHz) δ 7.24 (s, 1H), 5.78 (d, J=8.7 Hz, 1H), 5.3 (m, 4H), 5.13 (t, J=9.3 Hz, 1H), 5.04 (t, J=9.6 Hz, 1H), 4.92 (t, J=8.1 Hz, 1H), 4.53 (d, J=7.9 Hz, 1H), 4.49 (d, J=12.0 Hz, 1H), 4.36 (dd, J=12.5 Hz, J=4.2 Hz, 1H), 4.0-4.1 (m, 3H), 3.9 (m, 3H), 3.8 (m, 1H), 3.6 (m, 1H), 3.5 (m, 3H), 2.4 (s, 1H), 2.3 (s, 1H), 2.2 (m, 2H), 2.2 (m, 5H), 2.08 (3H), 2.07 (s, 3H), 2.02 (m, 3H), 2.01 (s, 3H), 1.99 (s, 3H), 1.96 (s, 3H), 1.83 (s, 3H), 0.7-1.7 (m, 25H); ¹³C NMR (CDCl₃, 100 Hz) δ 170.7, 170.4, 170.4, 169.7, 169.5, 169.2 (2 carbons), 143.3, 141.8 (2 carbons), 127.0, 122.1 (2 carbons), 101.0, 85.9, 85.0, 76.2, 76.1, 73.0, 72.3, 72.0, 71.8, 70.8, 70.3, 67.9, 61.9, 61.7, 52.2, 49.5, 42.0, 41.7, 39.7, 38.3, 38.2, 36.7, 33.1, 32.1, 31.6, 31.3, 29.9 (2 carbons), 29.6, 29.2, 24.8, 22.9, 21.0, 20.9, 20.8 (2 carbons), 20.6, 20.4, 19.2, 18.9, 14.3, 13.7, 11.0; HRESI/APCI Calcd for C₅₆H₇₉N₄O₁₉ m/e 1111.5339; measured m/e 1111.5322.

### Example 2k

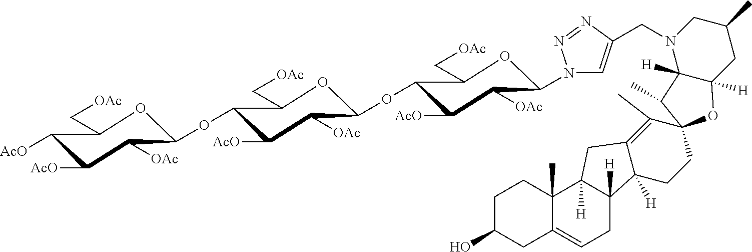

N-(1-(4-O-(4-O-(2,3,4,6-Tetra-O-acetyl-β-D-glucopyranosyl)-2,3,6-tri-O-acetyl-β- D-glucopyranosyl)-2,3,6-tri-O-acetyl-β-D-glucopyranosyl))-1H-1,2,3-triazol-4-yl)methylcyclopamine (2k). The general procedure for the 1,3-dipolar cycloaddition of propargylcyclopamine and an alkyl azide was used to prepare Example 2k. ¹H NMR (CDCl₃, 400 MHz) δ 7.70 (s, 1H), 5.87 (d, J=9.2 Hz, 1H), 5.3-5.5 (m, 6H), 5.06 (t, J=9.8 Hz, 1H), 4.86 (dd, J=10.5 Hz, J=4.0 Hz, 1H), 4.76 (dd, J=10.3 Hz, J=3.9 Hz, 1H), 4.49 (d, J=12.0 Hz, 2H), 4.36 (dd, J=12.4 Hz, J=4.3 Hz, 1H), 3.9-4.3 (m, 10H), 3.5 (m, 3H), 3.6 (m, 3H), 2.80 (d, J=9.1 Hz, 1H), 2.6 (m, 2H), 2.4 (m, 2H), 2.15 (s, 6H), 2.09 (s, 3H), 2.06 (s, 3H), 2.04 (s, 3H), 2.03 (s, 3H), 2.01 (s, 3H), 2.01 (s, 3H), 2.00 (s, 3H), 2.9 (m, 2H), 1.83 (s, 3H), 0.8-1.8 (m, 27H); ¹³C NMR (CDCl₃, 100 Hz) δ 170.9, 170.8, 170.8, 170.6 (2 carbons), 170.1, 169.9 (2 carbons), 169.7, 169.3, 143.3, 141.8, 131.1, 126.8, 122.1, 121.6, 96.3, 95.9, 85.6, 85.1, 75.6, 74.9, 73.9, 73.7, 72.6, 72.0, 71.8, 71.3, 70.7, 70.3, 70.1, 69.5, 69.5, 68.7, 68.1, 62.9, 62.4, 61.6, 61.1, 52.2, 49.9, 49.5, 42.0, 41.7, 39.6, 38.3, 38.1, 36.7, 33.0, 31.6, 31.3, 29.9, 29.2 (2 carbons), 24.8, 21.1, 21.0 (2 carbons), 21.0, 20.9, 20.8 (3 carbons), 20.4, 19.2, 18.9, 13.7, 11.0; HRESI/APCI Calcd for C₆₈H₉₅N₄O₂₇ ([MH]⁺) m/e 1399.6184; measured m/e 1399.6160.

General procedure for hydrolysis for Examples 3a-3k. To a solution of starting material (Examples 2a-2k; 0.04 g) in MeOH, two drops of water and K₂CO₃ (catalytic amount) were added. The reaction mixture was stirred vigorously at room temperature for two hours. After completion of the reaction as determined by TLC (eluted with CH₂Cl₂/MeOH=4/1), the reaction was quenched by the addition of Amberlite IR-120 (H⁺) and stirred for another several minutes. The reaction mixture was filtered through Celite and the residue was washed with MeOH. After the removal of the solvent, the product was purified by flash column chromatography (eluted from CH₂Cl₂ to CH₂Cl₂/MeOH=4/1). The products were obtained as white solids.

### Example 3a

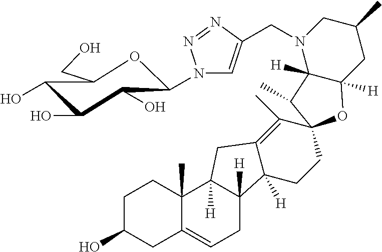

N-(1-(β-D-Glucopyranosyl)-1H-1,2,3-triazol-4-yl)methylcyclopamine (3a). The general procedure for the hydrolysis was used to prepare Example 3a. ¹H NMR (CD₃OD, 400 MHz) δ 8.16 (s, 1H), 5.60 (d, J=9.2 Hz, 1H), 5.40 (s, 1H), 4.00 (d, J=14.2 Hz, 1H), 3.89 (t, J=9.1 Hz, 1H), 3.88 (s, 1H), 3.7 (m, 1H), 3.4-3.6 (m, 8H), 2.92 (m, 1H), 2.7 (m, 1H), 2.2-2.4 (m, 6H), 2.0 (m, 2H), 0.9-1.8 (m, 24H); ¹³C NMR (CD₃OD, 100 Hz) δ 143.1 (2 carbons), 141.9, 126.6, 123.6, 121.5, 88.4, 85.5, 80.0, 77.3, 74.0, 72.8, 71.3, 70.5, 69.7, 61.3, 61.2, 52.2, 49.3, 48.9, 42.2, 41.4, 39.5, 38.3, 37.6, 36.5, 32.5, 30.8, 29.6, 29.1, 28.7, 24.5, 18.1, 17.8, 12.6, 10.0; HRESI/APCI Calcd for C₃₆H₅₅N₄O₇ ([MH]⁺) m/e 655.4071; measured m/e 655.4075.

### Example 3b

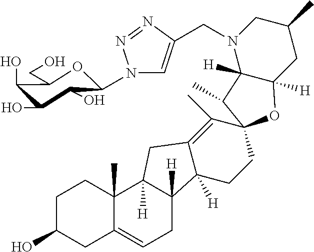

N-(1-(D-galactopyranosyl)-1H-1,2,3-triazol-4-yl)methylcyclopamine (3b). The general procedure for the hydrolysis was used to prepare Example 3b. ¹H NMR (CD₃OD, 400 MHz) δ 8.21 (s, 1H), 5.57 (d, J=9.1 Hz, 1H), 5.40 (s, 1H), 4.15 (t, J=9.3 Hz, 1H), 4.0 (m, 2H), 3.6-3.9 (m, 5H), 3.4-3.5 (m, 3H), 2.93 (d, J=8.0 Hz, 1H), 2.70 (t, J=7.1 Hz, 1H), 2.2-2.4 (7H), 2.0 (m, 2H), 0.9-1.9 (m, 24H); ¹³C NMR (CD₃OD, 100 Hz) δ 143.4, 143.1, 141.9, 126.6, 123.0, 121.5, 89.0, 85.5, 78.8, 74.1, 74.0, 71.3, 70.5, 70.3, 69.2, 61.3, 61.2, 52.2, 49.4, 48.9, 48.2, 42.2, 41.3, 39.6, 38.2, 37.6, 36.5, 32.5, 30.8, 29.1, 28.7, 24.5, 18.1, 17.7, 12.6, 9.9; HRESI/APCI Calcd for C₃₆H₅₅N₄O₇([MH]⁺) m/e 655.4071; measured m/e 655.4081.

### Example 3c

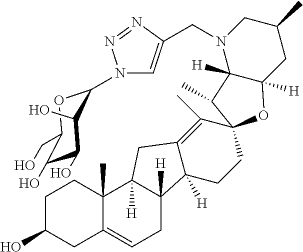

N-(1-(α-D-Mannopyranosyl)-1H-1,2,3-triazol-4-yl)methylcyclopamine (3c). The general procedure for the hydrolysis was used to prepare Example 3c. ¹H NMR (CD₃OD, 400 MHz) δ 8.22 (s, 1H), 6.02 (d, J=1.1 Hz, 1H), 5.40 (s, 1H), 4.14 (s, 1H), 4.0 (d, J=14.3 Hz, 1H), 3.94 (dd, J=12.1 Hz, J=2.0 Hz, 1H), 3.7-3.8 (m, 3H), 3.4-3.5 (m, 5H), 2.91 (d, J=8.5 Hz, 1H), 2.70 (t, J=7.1 Hz, 1H), 2.2-2.4 (6H), 2.0 (m, 2H), 0.9-1.9 (m, 25H); ¹³C NMR (CD₃OD, 100 Hz) δ 143.2, 142.4, 141.9, 126.6, 124.0, 121.5, 87.1, 85.5, 80.4, 74.0, 73.8, 71.3, 71.1, 70.4, 66.6, 61.4, 61.3, 52.2, 49.4, 48.9, 48.4, 42.2, 41.4, 39.6, 38.3, 37.6, 36.5, 32.5, 30.8, 29.0, 28.7, 24.5, 18.1, 17.8, 12.6, 10.0; HRESI/APCI Calcd for C₃₆H₅₅N₄O₇ ([MH]⁺) m/e 655.4071; measured m/e 655.4083.

### Example 3d

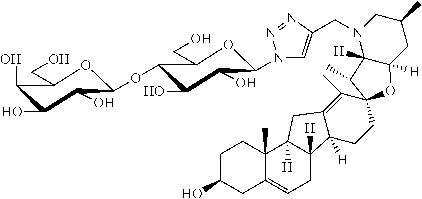

N-(1-(4-O-(β-D-Galactopyranosyl)-β-D-glucopyranosyl)-1H-1,2,3-triazol-4-yl)methylcyclopamine (3d). The general procedure for the hydrolysis was used to prepare Example 3d. ¹H NMR (CD₃OD, 400 MHz) δ 8.15(s, 1H), 5.64 (d, J=9.2 Hz, 1H), 5.39 (s, 1H), 4.43 (d, J=7.7 Hz, 1H), 3.3-4.0 (m, 17H), 2.9 (m, 1H), 2.7 (m, 1H), 2.2-2.3 (m, 6H), 2.0 (m, 3H), 0.9-1.8 (m, 24H); ¹³C NMR (CD₃OD, 100 Hz) δ 143.2, 143.1, 142.1, 126.3, 123.5, 121.5, 103.9, 88.1, 85.5, 78.6, 78.4, 76.0, 75.7, 74.0, 73.7, 72.5, 71.4, 71.3, 70.5, 69.1, 61.3 (2 carbons), 60.4, 52.3, 49.4, 48.9, 42.3, 41.4, 39.6, 38.1, 37.6, 36.5, 32.5, 30.9 (2 carbons), 29.1, 28.7, 24.5, 18.1, 17.8, 12.6, 9.9; HRESI/APCI Calcd for C₄₂H₆₅N₄O₁₂ ([MH]⁺) m/e 817.4599; measured m/e 817.4568.

### Example 3e

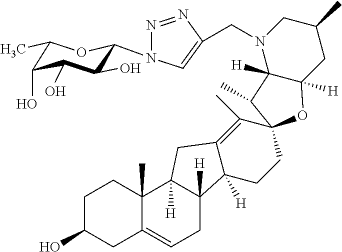

N-(1-(β-L-Fucopyranosyl)-1H-1,2,3-triazol-4-yl)methylcyclopamine (3e). The general procedure for the hydrolysis was Used to prepare Example 3e. ¹H NMR (CD₃OD, 400 MHz) δ 8.19 (s, 1H), 5.54 (d, J=9.2 Hz, 1H), 5.40 (s, 1H), 4.06 (t, J=9.3 Hz, 1H), 4.0 (m, 2H), 3.76 (d, J=3.1 Hz, 1H), 3.6-3.7 (m, 2H). 3.4-3.5 (m, 4H), 2.93 (d, J=8.7 Hz, 1H), 2.69 (t, J=7.2 Hz, 1H), 2.2-2.3 (m, 7H), 2.0 (m, 2H), 0.9-1.8 (m, 26H); ¹³C NMR (CD₃OD, 100 Hz) δ 143.4, 143.1, 141.9, 126.2, 122.7, 121.5, 89.0, 85.5, 74.3, 74.1, 74.0, 71.8, 71.3, 70.4, 70.1, 61.3, 52.2, 49.4, 48.9, 42.2, 41.4, 39.6, 38.3, 37.6, 36.5, 32.5, 30.8, 29.9, 29.1, 28.7, 24.5, 18.1, 17.8, 15.6, 12.6, 10.0; HRESI/APCI Calcd for C₃₆H₅₅N₄O₆ ([MH]⁺) m/e 639.4122; measured m/e 639.4113.

### Example 3f

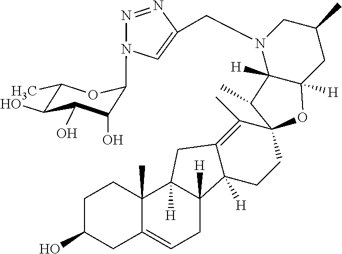

N-(1-(α-L-Rhamnopyranosyl)-1H-1,2,3-triazol-4-yl)methylcyclopamine (3f). The general procedure for the hydrolysis was used to prepare Example 3f. ¹H NMR. (CD₃OD, 400 MHz) δ 8.10 (s, 1H), 5.98 (d, J=1.1 Hz, 1H), 5.40 (s, 1H), 4.10 (dd, J=3.0 Hz, J=1.1 Hz, 1H), 4.00 (d, J=14.2 Hz, 1H), 3.70 (dd, J=9.1 Hz, J=3.2 Hz, 1H), 3.65 (s, 1H), 3.4-3.6 (m, 6H), 2.90 (d, J=8.7 hz, 1H), 2.67 (t, J=7.2 Hz, 1H), 2.2-2.3 (m, 7H), 2.0 (m, 2H), 0.9-1.8 (m, 26H); ¹³C NMR (CD₃OD, 100 Hz) δ 143.1, 142.5, 141.9, 126.6, 123.8, 121.5, 87.0, 85.5, 75.8, 74.0, 73.6, 71.9, 71.3, 71.2, 70.5, 61.2, 52.2, 49.4, 48.9, 42.2, 41.4, 39.6, 38.3, 37.6, 36.5, 32.5, 30.8, 30.2, 29.0, 28.7, 24.5, 18.1, 17.8, 16.9, 12.6, 10.0; HRESI/APCI Calcd for C₃₆H₅₅N₄O₆ ([MH]⁺) m/e 639.4122; measured m/e 639.4118.

### Example 3g

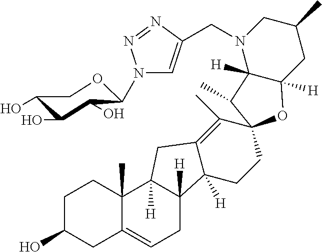

N-(1-(α-D-Xylopyranosyl)-1H-1,2,3-triazol-4-yl)methylcyclopamine (3g). The general procedure for the hydrolysis was used to prepare Example 3g. ¹H NMR (CD₃OD, 400 MHz) δ 8.11 (s, 1H), 5.50 (d, J=9.2 Hz, 1H), 5.40 (s, 1H), 4.03 (t, J=5.6 Hz, 1H), 4.0 (d, J=4.2 Hz, 1H), 3.92 (t, J=9.1 Hz, 1H), 3.7 (m, 3H), 3.5 (m, 5H), 2.91 (d, J=9.2 Hz, 1H), 2.67 (t, J=7.1 Hz, 1H), 2.2-2.3 (m, 6H), 2.0 (m, 2H), 0.9-1.9 (m, 24H); ¹³C NMR (CD₃OD, 100 Hz) δ 143.2 (2 carbons), 142.0, 126.6, 123.5, 121.5. 89.0, 85.5, 77.5, 74.0, 72.7, 71.3, 70.4, 69.5, 68.7, 61.3, 52.3, 48.8, 48.9, 42.3, 41.4, 39.6, 38.2, 3776, 36.5, 32.5, 30.9, 30.8, 29.1, 28.7, 24.5, 18.1, 17.7, 12.6, 9.9; HRESI/APCI Calcd for C₃₅H₅₃N₄O₆ ([MH]³⁰ ) m/e 625.3965; measured m/e 625.3956.

### Example 3h

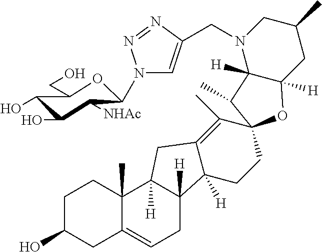

N-(1(2-Acetamido-2-deoxy-α-D-glucopyransoyl)-1H-1,2,3-triazol-4-yl)methylcyclopamine yl)methylcyclopamine (3h). The general procedure for the hydrolysis was used to prepare Example 3h. ¹H NMR (CD₃OD, 400 MHz) δ 8.12 (s, 1H), 5.78 (d, J=9.9 Hz, 1H), 5.39 (s, 1H), 4.26 (t, J=8.0 Hz, 1H), 3.92 (t, J=10.0 Hz, 2H), 3.7 (m, 1H), 3.4-3.7 (m, 9H), 2.82 (d, J=7.7 Hz, 1H), 2.7 (m, 1H), 2.1-2.3 (m, 6H), 1.9-2.0 (m, 2H), 0.9-1.8 (m, 26H); ¹³C NMR (CD₃OD, 100 Hz) δ 171.9, 143.1, 142.7, 141.9, 126.6, 123.0, 121.5, 87.0, 85.5, 80.1, 74.6, 74.0, 71.3, 70.2, 69.8, 61.1, 60.8, 55.4, 52.2, 48.9, 48.4, 42.2, 41.4, 39.5, 38.3, 37.5, 36.5, 32.5, 30.8, 2.96, 29.1, 28.7, 24.5, 21.6, 18.1, 17.8, 12.7, 10.0; HRESI/APCI Calcd for C₃₈H₅₈N₅O₇ ([MH]⁺) m/e 696.4336 ; measured m/e 696.4338.

### Example 3i

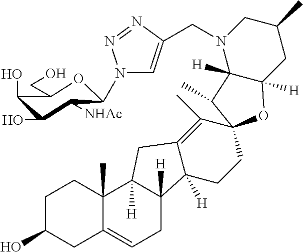

N-(1-(2-Acetamido-2-deoxy-α-D-galactopyranosyl)-1H-1,2,3-triazol-4-yl)methylcyclopamine (3i). The general procedure for the hydrolysis was used to prepare Example 3i. ¹H NMR (CD3OD, 400 MHz) δ 8.12 (s, 1H), 5.77 (d, J=9.9 Hz, 1H), 5.39 (s, 1H), 4.26 (t, J=10.0 Hz, 1H), 3.9 (m, 2H), 3.6-3.8 (m, 7H), 3.4-3.5 (m, 2H), 2.9 (m, 1H), 2.8 (m, 1H), 2.1-2.3 (m, 5H), 2.0 (m, 5H), 0.9-1.9 (m, 25H); ¹³C NMR (CD₃OD, 100 Hz) δ 171.9, 143.1, 142.6, 141.9, 126.6, 123.0, 121.5, 87.0, 85.5, 80.1, 74.6, 74.0, 71.3, 70.2, 69.3, 68.4, 61.2, 60.7, 55.4, 52.2, 48.9, 48.7, 42.2, 41.4, 39.5, 38.3, 37.5, 36.5, 32.5, 30.8, 29.0, 28.7. 24.5, 21.6, 18.1, 17.8, 12.7, 10.0; HRESI/APCI Calcd for C₃₈H₅₈N₅O₇ ([MH]⁺) m/e 696.4336; measured m/e 696.4333.

### Example 3j

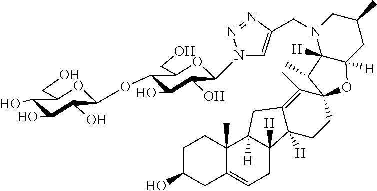

N-(1-(4-O-(β-D-Glucopyranosyl)-β-D-glucopyranosyl)-1H-1,2,3-triazol-4-yl)methylcyclopamine (3j). The general procedure for the hydrolysis was used to prepare Example 3j. ¹H NMR (CD₃OD, 400 MHz) δ 8.15 (s, 1H), 5.64 (d, J=9.2 Hz, 1H), 5.40 (s, 1H), 4.48 (d, J=7.8 Hz, 1H), 3.3-4.0 (m, 17H), 2.9 (m 1H), 2.7 (m, 1H), 2.2-2.3 (m, 7H), 2.0 (m, 3H), 0.7-1.8 (m, 23H); ¹³C NMR (CD₃OD, 100 Hz) δ 143.3, 143.1, 142.0, 126.6, 123.5, 121.5, 103.4, 88.1, 85.5, 78.5. 78.4, 77.0, 76.7, 75.7, 74.0, 73.7, 72.6, 71.3, 70.5, 70.2, 61.3 (2 carbons), 60.3, 52.3, 49.4, 48.9, 42.3. 41.4, 39.6, 38.3, 37.6, 36.5, 32.5, 30.8 30.7, 29.1, 28.7, 24.5, 18.1, 17.8, 12.6, 9.9; HRESI/APCI Calcd for C₄₈H₇₅N₄O₁₇ ([MH]⁺) m/e 817.4599; measured m/e 817.4586.

### Example 3k

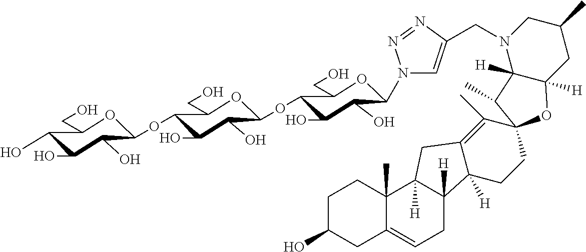

N-(1-(4-O-(4-O-(β-Glucopyranosyl)-β-D-glucopyranosyl)-β-D-glucopyranosyl))- 1H-1,2,3-triazol-4-yl)methylcyclopamine (3k). The general procedure for the hydrolysis was used to prepare Example 3k. ¹H NMR (CD₃OD, 400 MHz) δ 8.17 (s, 1H), 5.64 (d, J=9.0 Hz, 1H), 5.40 (s, 1H), 5.26 (d, J=3.7 Hz, 1H), 5.17 (d, J=3.8 Hz, 1H), 3.5-4.0 (m, 24H), 2.89 (d, J=8.0 Hz, 1H), 2.7 (m, 1H), 2.0-2.3 (m, 8H), 0.7-1.8 (m, 24H); ¹³C NMR (CD₃OD, 100 Hz) δ 143.3, 143.1, 141.9, 126.6, 123.6, 121.5. 101.8, 101.6, 88.2, 85.5, 80.2, 79.2, 78.5, 77.1, 74.0, 73.9, 73.8, 73.6, 73.1, 72.6, 72.5, 72.3, 71.3, 70.5, 70.3, 61.5, 61.3, 60.9, 60.7, 52.2, 49.4, 48.9, 42.3, 41.4, 39.6, 38.3, 37.6, 36.5, 32.5, 30.8, 29.6, 29.1, 28.7, 24.5, 18.1, 17.8, 12.6, 10.0; HRESI/APCI Calcd for C₄₅H₇₅N₄O₁₇ ([MH]⁺) m/e 979.5127; measured m/e 979.5109.

### Example 3l

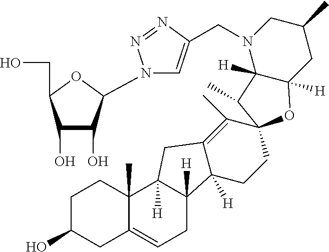

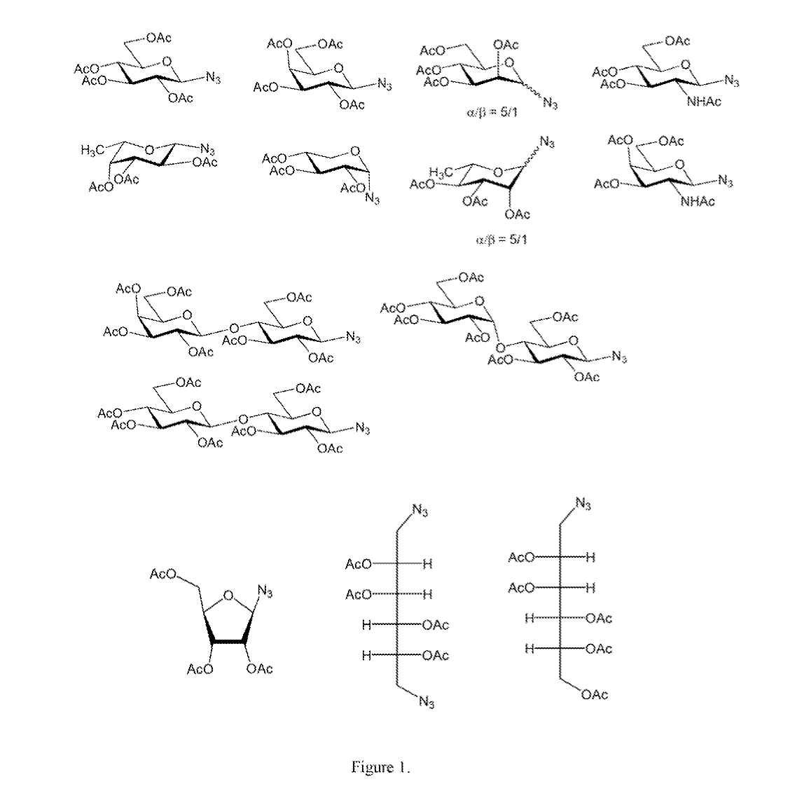

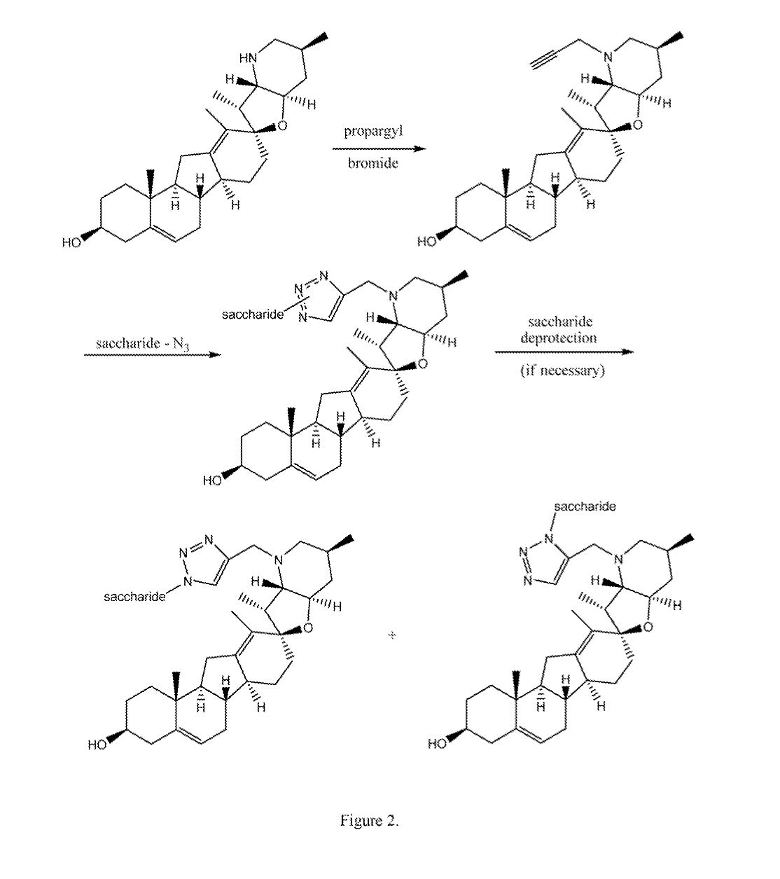

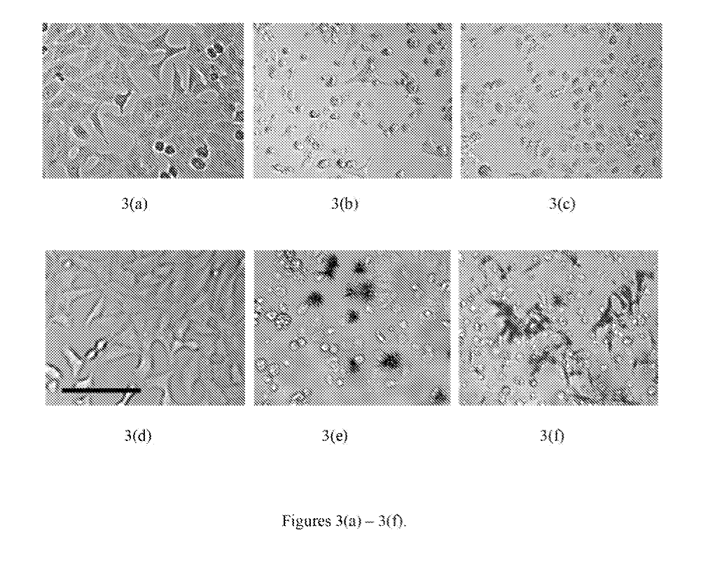

N-(1-(ribofuranosyl)-1H-1,2,3-triazol-4-yl)methylcyclopamine (3l). Example 3l was synthesized using the general procedures described above, using the corresponding alkyl azide shown in FIG. 1. ¹H NMR (CD₃OD, 400 MHz) δ 8.24 (s, 1H), 6.04 (d, J=3.9 Hz, 1H), 5.4 (m, 1H), 5.51 (t, J=4.3 Hz, 1H), 4.32 (t, J=5.0 Hz, 1H), 4.2 (m, 1H), 4.0 (d, J=14.2 Hz, 1H), 3.82 (dd, J=15.3, 3.2 Hz, 1H), 3.71 (dd, J=15.0, 4.2 Hz, 1H), 3.4-3.5 (m, 4H), 2.7 (m, 1H), 2.6 (m, 1H), 0.9-2.3 (m, 33H); ¹³C NMR (CD₃OD, 100 MHz) δ 143.1, 141.9, 126.6, 123.0, 121.5, 93.2, 86.0, 85.5, 75.9, 74.0, 71.3, 70.7, 70.4, 61.6, 61.3, 52.2, 52.2, 49.3, 48.9, 42.2, 41.4, 39.5, 38.3, 37.6, 36.5, 32.5, 30.8, 29.6, 28.7, 24.5, 18.1, 17.8, 13.3, 12.6, 10.0; HRESI Calcd for C₃₅H₅₃N₄O₆ ([MH]⁺) m/e 625.3960; measured m/e 625.3967.

### Example 3m

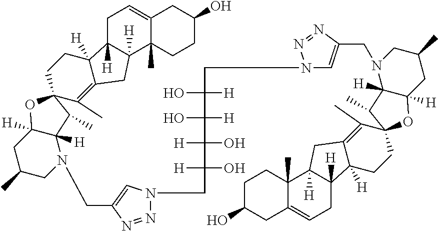

N-(1-(mannitol)-1H-1,2,3-triazol-4-yl)methylcyclopamine dimer (3m). Example 3m was synthesized using the general procedures described above, using the corresponding alkyl azide shown in FIG. 1. ¹H NMR (CD₃OD, 300 MHz) δ 7.92 (s, 2H), 5.37 (d, 2H), 4.77 (dd, J=13.7, 2.4 Hz, 2H), 4.45 (dd, J=14.1, 7.6 Hz, 2H), 4.9 (m, 4H), 3.6 (m, 4H), 3.3-3.4 (m, 6H), 2.9 (m, 2H), 2.6 (m, 2H), 0.8-2.3 (m, 66H); ¹³C NMR (CD3OD, 100 MHz) δ 143.2 (2 carbons), 141.9 (2 carbons), 126.6 (2 carbons), 125.4 (2 carbons), 121.5 (2 carbons), 85.5 (2 carbons), 74.0 (2 carbons), 71.3 (2 carbons), 70.5 (2 carbons), 70.3 (2 carbons), 70.1 (2 carbons), 61.3 (2 carbons), 53.8 (2 carbons), 52.2 (2 carbons), 49.4 (2 carbons), 48.2 (2 carbons), 42.2 (2 carbons), 41.4 (2 carbons), 39.6 (2 carbons), 38.3 (2 carbons), 37.6 (2 carbons), 36.5 (2 carbons), 32.5 (2 carbons), 30.8 (4 carbons), 29.6 (2 carbons), 29.1 (2 carbons), 28.7 (2 carbons), 24.5 (2 carbons), 18.2 (2 carbons), 17.8 (2 carbons), 12.6 (2 carbons), 10.0 (2 carbons): HRESI Calcd for C₆₆H₉₈N₈O₈Na ([M+Na]³⁰ ) m/e 1153.7400; measured m/e 1153.7402.

### Example 3n

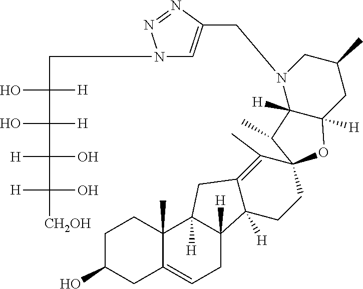

N-(1-(mannitol)-1H-1,2,3-triazol-4-yl)methylcyclopamine (3n). Example 3n was synthesized using the general procedures described above, using the corresponding alkyl azide shown in FIG. 1. ¹H NMR (CD₃OD, 400 MHz) δ 7.95 (s, 1H), 5.4 (m, 1H), 4.8 (m,1H), 4.5 (m 1H), 4.0 (m, 2H), 3.9 (m, 3H), 3.6 (m, 6H), 3.3 (m, 3H), 2.9 (m, 1H), 2.8 (m, 1H), 0.8-2.3 (m, 30H): ¹³C NMR (CD₃OD, 100 MHz) δ 143.2, 142.7, 141.9, 126.6, 125.4, 121.5, 85.5 , 74.0, 71.7, 71.3, 70.9, 70.5, 70.2, 69.8, 63.9, 61.3, 53.9, 52.2, 49.4, 48.9, 42.2, 41.4, 39.6, 38.2, 37.6, 36.5, 32.5, 29.6, 29.1, 28.7, 24.5, 18.1, 17.8, 13.2, 12.6, 10.9; HRESI Calcd for C₃₆H₅₇N₄O₇ ([MH]⁺) m/e 657.42222; measured m/e 657.4227.

LC-MS profile for Compound 3f: Mass spectra were obtained by flow injection electrospray mass spectrometry. Samples were dissolved in methanol (˜0.1 mg/mL) and 5 uL injected for analysis using a HP1100 binary solvent delivery system coupled to a Finnigan LCQ mass spectrometer. Solvent flow was 50% methanol and 0.1% formic acid at a flow rate of 0.5 mL/min. Ionization was achieved with a standard electrospray ionization source. Ionization parameters were optimized by first tuning on a solution of resperine (MH⁺=609). A Betasil C18 reverse phase HPLC column (100×2.1 mm) was used inline with the above HPLC pump and mass spectrometer. The solvent flow was a gradient using methanol (A) and 20 mM ammonium acetate (B), starting with 60% A for 1.0 min, a linear increase to 100% A from 1 to 15 min, followed by 100% A from 15 to 25 min. The flow rate was 0.3 mL/min. Peak area measurement from the base peak ion chromatogram was used to assess purity. The purity of 3f was calculated to be 96.3%, which consists of 89% α-3f (the a anomer of 30 and 11% β-3f (the β anomer of 3f).

The yields of Examples 2a-2k and 3a-3k and ratios are shown below. With the exception of Example 3f, the α/β ratio was calculated based on the integration of the anomeric proton signal from ¹H NMR. The α/β ratio of Example 3f was calculated from LC-MS analysis (described above).

| example | yield (%) | α/β ratio | example | yield (%) | α/β ratio |
|---|---|---|---|---|---|
| 2a | 82 | β only | 3a | 84 | β only |
| 2b | 82 | β only | 3b | 84 | β only |
| 2c | 82 | α only | 3c | 84 | α only |
| 2d | 81 | β only | 3d | 68 | β only |
| 2e | 99 | β only | 3e | 60 | β only |
| 2f | 99 | α only | 3f | 60 | 89/11 |
| 2g | 99 | β only | 3g | 60 | β only |
| 2h | 82 | β only | 3h | 85 | β only |
| 2i | 99 | β only | 3i | 99 | β only |
| 2j | 81 | β only | 3j | 68 | β only |
| 2k | 80 | β only | 3k | 86 | β only |

The biological activity of the compounds of Formula I and Formula II was demonstrated by the MTS assay.

Experimental procedure for MTS assay. To determine the efficacy of the compounds to cancer cells, a 3-(4,5-dimethylthiazol-2-yl)-5-(3-carboxymethoxyphenyl)-2-(4- sulfophenyl)-2H-tetrazolium inner salt (MTS) assay was used. A549 cells (ATCC, Manassas, Va.), representing a human lung carcinoma cell line, were plated on flat-bottomed 96-well plates in the presence of various concentrations of each compound. After incubation for up to 48 h at 37° C., the percentage of cell proliferation was determined with the MTS assay (Promega CellTiter 96 Aqueous Assay. Madison, Wis.), following the manufacturer's instruction.

The response of the A549 cells to the compounds tested indicates the potential for the compound to act as an anticancer drug. If the number of A549 cells that proliferate (or grow) in the presence of a compound is reduced in comparison to the number of A549 cells that proliferate in the absence of the compound, then the compound is determined to exhibit potential anticancer activity.

TABLE 1
Anticancer activity of selected saccharide-cyclopamine
conjugates against lung cancer.

| Compound | IC₅₀ (μM) |
|---|---|
| 1, cyclopamine | 49 |
| 3a | 144 |
| 3d | 183 |
| 3f | 33 |
| 3k | 96 |

The results shown in Table I show that cyclopamine and Examples 3a, 3d, 3f, and 3k reduced the proliferation of A549 cells, exhibiting potential anticancer activity. Example 3f reduced the proliferation of A549 cells more than cyclopamine, suggesting that Example 3f is a more active anticancer compound than cyclopamine against human lung carcinoma.

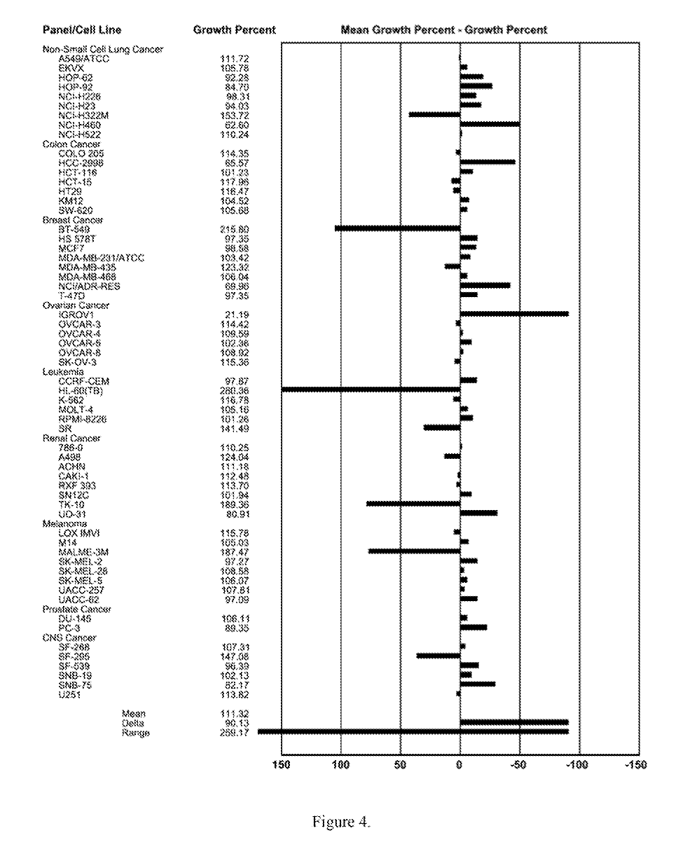

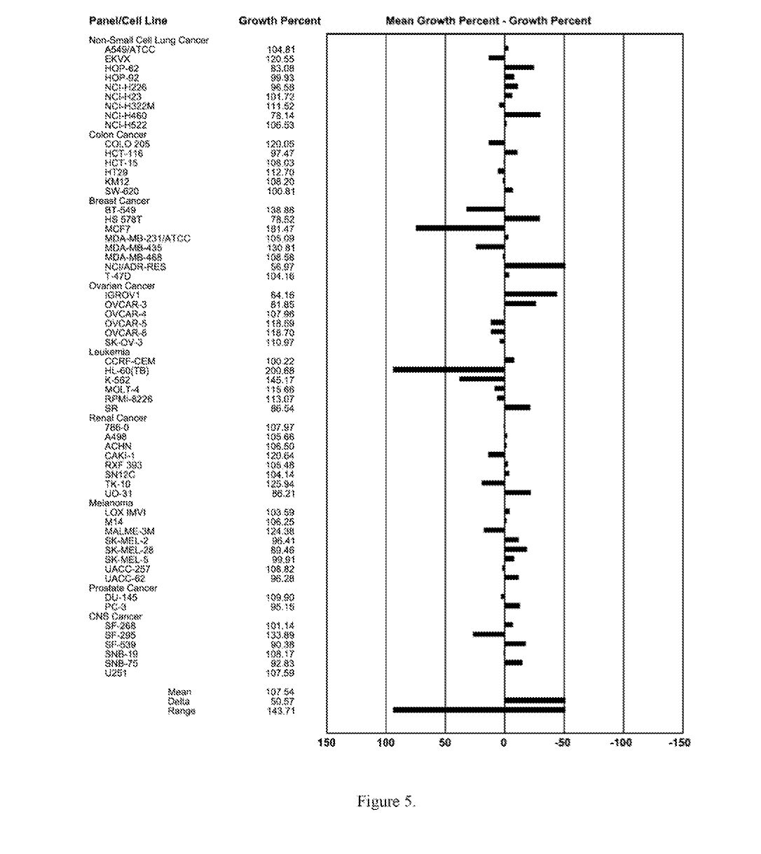

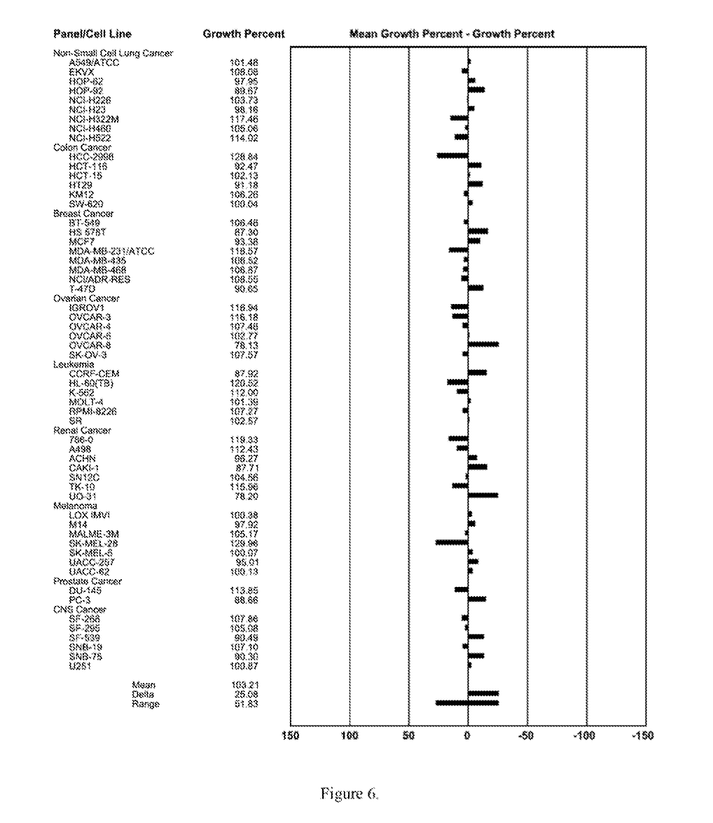

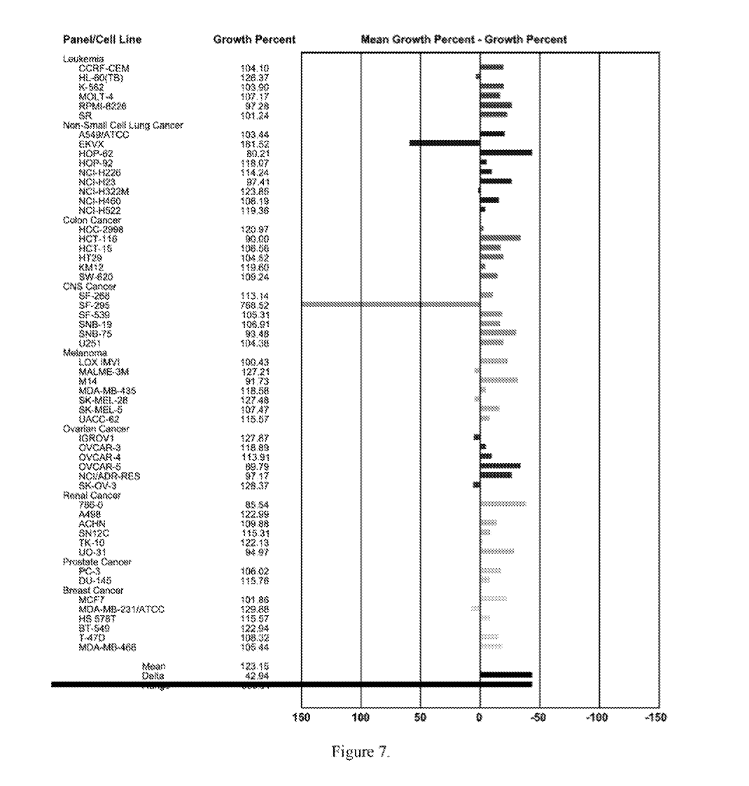

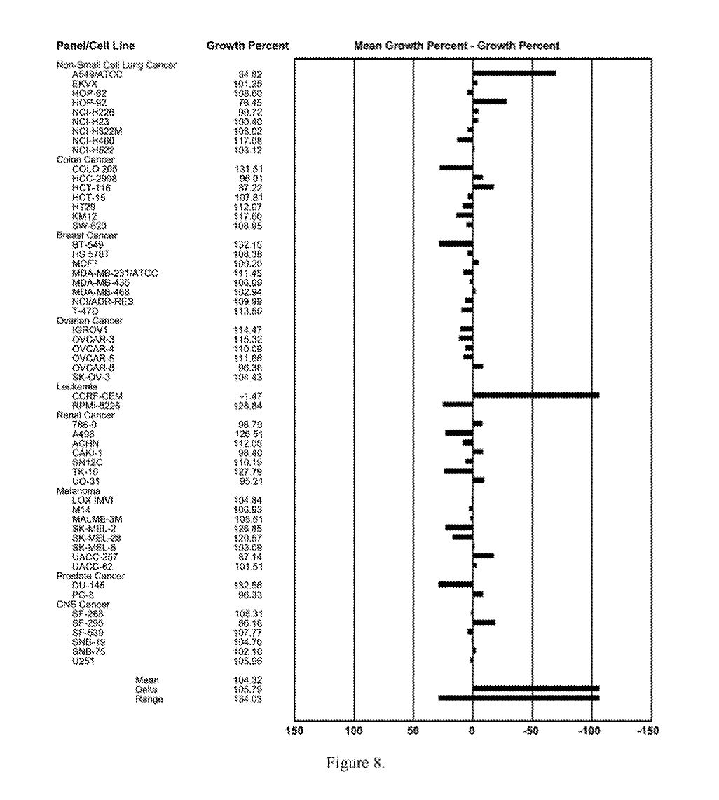

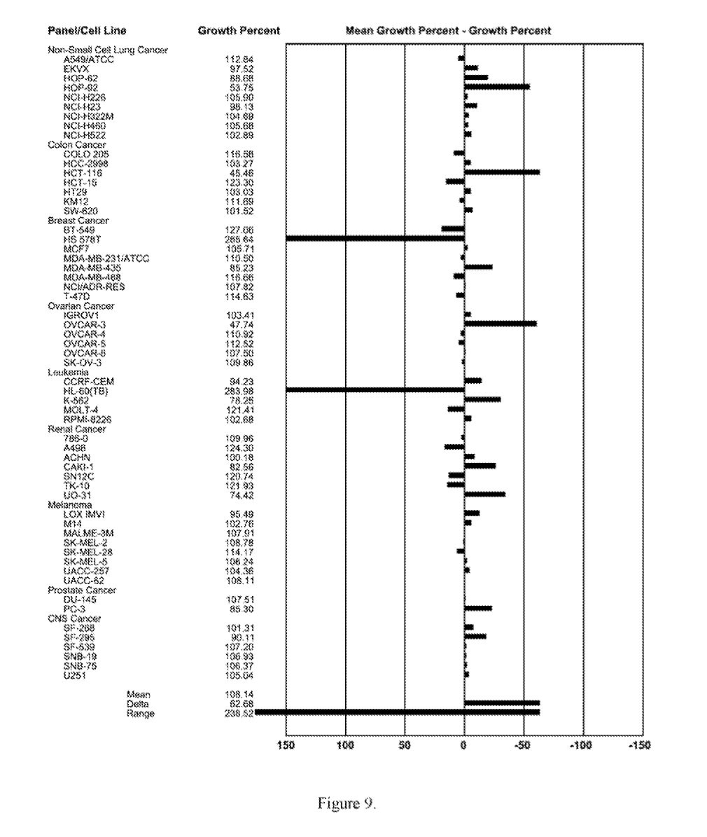

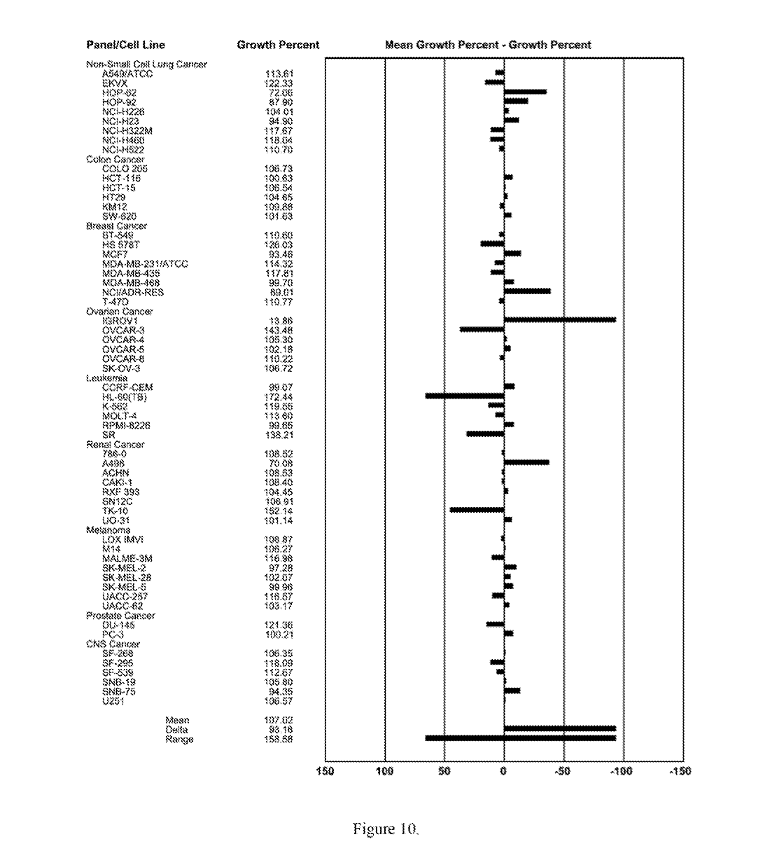

The results shown in FIGS. 4-10 show the anticancer activity of the compounds of Examples 3a (FIG. 4), 3d (FIG. 5), 3e (FIG. 6), 3f (FIG. 7), 3h (FIG. 8), 3i (FIGS. 9), and 3k (FIG. 10), in a 60-cell line panel. A negative value for the growth percentage (i.e. a bar extending to the right of zero) is an indication of anticancer activity.

All publications, including but not limited to patents and patent applications, cited in this specification are herein incorporated by reference as if each individual publication were specifically and individually indicated to be incorporated by reference herein and as though fully set forth.

It will be obvious to those having skill in the art that many changes may be made to the details of the above-described embodiments without departing from the underlying principles of the invention. The scope of the present invention should, therefore, be determined only by the following claims.

Modifications and improvements of the embodiments specifically disclosed herein are within the scope of the following claims. Without further elaboration, it is believed that one skilled in the area can, using the preceding description, utilize the present disclosure to its fullest extent. Therefore the Examples herein are to be construed as merely illustrative and not a limitation of the scope of the present invention in any way, although the specifics recited herein may include independently patentable subject matter. The embodiments disclosed in which an exclusive property or privilege is claimed are defined as follows.

1. A compound of Formula I: 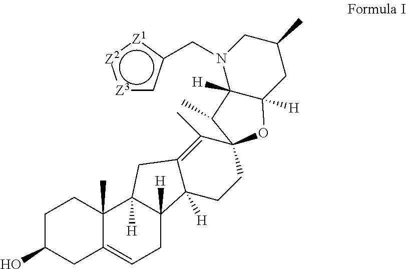
wherein:
Z¹-Z²-Z³ is N(R¹)-N═N or N═N-N(R¹); and
R¹ is at least one heterocycle optionally substituted with up to 7 substituents selected from the group consisting of H, OH, OR², SH, SR², N(R²)₂, alkyl, and halogen; or
R¹ is at least one straight-chain saccharide optionally substituted with up to 7 substituents selected from the group consisting of H, OH, OR², SH, SR², N(R²)₂, alkyl, and halogen; and
R² is independently hydrogen, alkyl, aryl, acyl, aralkyl, or cycloalkyl; and pharmaceutically acceptable salts, hydrates, tautomers, dimers, solvates and complexes thereof.

2. The compound according to claim 1, wherein R¹ is a saccharide.

3. The compound according to claim 2, wherein R¹ is a pyranose.

4. The compound according to claim 3, wherein R¹ is an α-pyranose.

5. The compound according to claim 4, wherein R¹ is an α-rhamnose.

6. The compound according to claim 1, wherein the compound is selected from the group consisting of:
N-(1-(2,3,4,6-tetra-O-acetyl-β-D-glucopyranosyl)-1H-1,2,3-triazol-4-yl)methylcyclopamine;
N-(1-(2,3,4,6-tetra-O-acetyl-(β-D-galactopyranosyl)-1H-1,2,3-triazol-4-yl)methylcyclopamine;
N-(1-(2,3,4,6-tetra-O-acetyl-α-D-mannopyranosyl)-1H-1,2,3-triazol-4-yl)methylcyclopamine;
N-(1-(4-O-(2,3,4,6-tetra-O-acetyl-β-D-galactopyranosyl)-2,3,6-tri-O-acetyl-β- D-glucopyranosyl)-1H-1,2,3-triazol-4-yl)methylcyclopamine;
N-(1-(2,3,4-tri-O-acetyl-β-L-fucopyranosyl)-1H-1,2,3-triazol-4-yl)methylcyclopamine;
N-(1-(2,3,4-tri-O-acetyl-α-L-rhamnopyranosyl)-1H-1,2,3-triazol-4-yl)methylcyclopamine;
N-(1-(2,3,4-tri-O-acetyl-α-D-xylopyranosyl)-1H-1,2,3-triazol-4-yl)methylcyclopamine;
N-(1-(2-acetamido-3,4,6-tri-O-acetyl-2-deoxy-α-D-glucopyranosyl)-1H-1,2,3- triazol-4-yl)methylcyclopamine;
N-(1-(2-acetamido-3,4,6-tri-O-acetyl-2-deoxy-α-D-galactopyranosyl)-1H-1,2,3- triazol-4-yl)methylcyclopamine;
N-(1-(4-O-(2,3,4,6-tetra-O-acetyl-β-D-glucopyranosyl)-2,3,6-tri-O-acetyl-β-D- glucopyranosyl)-1H-1,2,3-triazol-4-yl)methylcyclopamine;
N-(1-(4-O-(4-O-(2,3,4,6-tetra-O-acetyl-β-D-glucopyranosyl)-2,3,6-tri-O-acetyl- β-D-glucopyranosyl)-2,3,6-tri-O-acetyl-β-D-glucopyranosyl))-1H-1,2,3-triazol-4-yl)methylcyclopamine;
N-(1-(β-D-glucopyranosyl)-1H-1,2,3-triazol-4-yl)methylcyclopamine;
N-(1-(D-galactopyranosyl)-1H-1,2,3-triazol-4-yl)methylcyclopamine;
N-(1-(α-D-mannopyranosyl)-1H-1,2,3-triazol-4-yl)methylcyclopamine;
N-(1-(4-O-(β-D-galactopyranosyl)-β-D-glucopyranosyl)-1H-1,2,3-triazol-4-yl)methylcyclopamine;
N-(1-(β-L-fucopyranosyl)-1H-1,2,3-triazol-4-yl)methylcyclopamine;
N-(1-(α-L-rhamnopyranosyl)-1H-1,2,3-triazol-4-yl)methylcyclopamine;
N-(1-(α-D-xylopyranosyl)-1H-1,2,3-triazol-4-yl)methylcyclopamine;
N-(1-(2-acetamido-2-deoxy-α-D-glucopyranosyl)-1H-1,2,3-triazol-4-yl)methylcyclopamine;
N-(1-(2-acetamido-2-deoxy-α-D-galactopyranosyl)-1H-1,2,3-triazol-4-yl)methylcyclopamine;
N-(1-(4-O-(β-D-glucopyranosyl)-β-D-glucopyranosyl)-1H-1,2,3-triazol-4-yl)methylcyclopamine;
N-(1-(4-O-(4-O-(β-D-glucopyranosyl)-β-D-glucopyranosyl)-β-D- glucopyranosyl))-1H-1,2,3-triazol-4-yl)methylcyclopamine;
N-(1-(ribofuranosyl)-1H-1,2,3-triazol-4-yl)methylcyclopamine;
N-(1-(mannitol)-1H-1,2,3-triazol-4-yl)methylcyclopamine dimer; and
N-(1-(mannitol)-1H-1,2,3-triazol-4-yl)methylcyclopamine.

7. The compound according to claim 1, wherein the compound is selected from the group consisting of:
N-(1-(2,3,4-tri-O-acetyl-α-L-rhamnopyranosyl)-1H-1,2,3-triazol-4-yl)methylcyclopamine; and
N-(1-(α-L-rhamnopyranosyl)-1H-1,2,3-triazol-4-yl)methylcyclopamine.

8. A compound of Formula II: 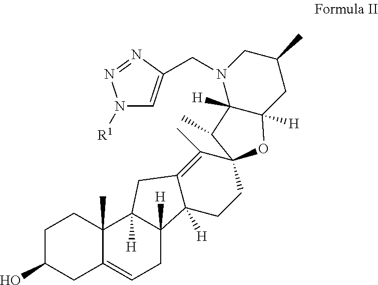
wherein:
R¹ is at least one heterocycle optionally substituted with up to 7 substituents selected from the group consisting of H, OH, OR², SH, SR², N(R²)₂, alkyl, and halogen; or
R¹ is at least one straight-chain saccharide optionally substituted with up to 7 substituents selected from the group consisting of H, OH, OR², SH, SR², N(R²)₂, alkyl, and halogen; and
R² is independently hydrogen, alkyl, aryl, acyl, aralkyl, or cycloalkyl; and pharmaceutically acceptable salts, hydrates, tautomers, dimers, solvates and complexes thereof.

9. The compound according to claim 8, wherein R¹ is selected from the group consisting of a saccharide, a pyranose, an α-pyranose, and an α-rhamnose.

10-12. (canceled)

13. The compound according to claim 8, wherein the compound is selected from the group consisting of:
N-(1-(β-D-glucopyranosyl)-1H-1,2,3-triazol-4-yl)methylcyclopamine;
N-(1-(D-galactopyranosyl)-1H-1,2,3-triazol-4-yl)methylcyclopamine;
N-(1-(α-D-mannopyranosyl)-1H-1,2,3-triazol-4-yl)methylcyclopamine;
N-(1-(4-O-(β-D-galactopyranosyl)-β-D-glucopyranosyl)-1H-1,2,3-triazol-4-yl)methylcyclopamine;
N-(1-(3-L-fucopyranosyl)-1H-1,2,3-triazol-4-yl)methylcyclopamine;
N-(1-(α-L-rhamnopyranosyl)-1H-1,2,3-triazol-4-yl)methylcyclopamine;
N-(1-(α-D-xylopyranosyl)-1H-1,2,3-triazol-4-yl)methylcyclopamine;
N-(1-(2-acetamido-2-deoxy-α-D-glucopyranosyl)-1H-1,2,3-triazol-4-yl)methylcyclopamine;
N-(1-(2-acetamido-2-deoxy-α-D-galactopyranosyl)-1H-1,2,3-triazol-4-yl)methylcyclopamine;
N-(1-(4-O-(β-D-glucopyranosyl)-β-D-glucopyranosyl)-1H-1,2,3-triazol-4-yl)methylcyclopamine;
N-(1-(4-O-(4-O-(β-D-glucopyranosyl)-β-D-glucopyranosyl)-β-D- glucopyranosyl))-1H-1,2,3-triazol-4-yl)methylcyclopamine;
N-(1-(ribofuranosyl)-1H-1,2,3-triazol-4-yl)methylcyclopamine;
N-(1-(mannitol)-1H-1,2,3-triazol-4-yl)methylcyclopamine dimer; and
N-(1-(mannitol)-1H-1,2,3-triazol-4-yl)methylcyclopamine.

14. The compound according to claim 8, wherein the compound is:
N-(1-(α-L-rhamnopyranosyl)-1H-1,2,3 -triazol-4-yl)methylcyclopamine.

15. The compound according to claim 8, wherein:
R¹ is at least one pyranose optionally substituted with up to 7 substituents selected from the group consisting of H, OH, OR², SH, SR², N(R²)₂, alkyl, and halogen; and
R² is independently hydrogen, alkyl, aryl, acyl, aralkyl, or cycloalkyl; and
pharmaceutically acceptable salts, hydrates, tautomers, solvates and complexes thereof.

16-19. (canceled)

20. A pharmaceutical composition comprising a compound according to claim 1 and a pharmaceutically acceptable diluent or excipient.

21. A method of treating cancer the method comprising administering to a subject in need of treatment an effective amount of pharmaceutical composition of claim 20.

22. A method according to claim 21, wherein the cancer is selected from the group consisting of pancreatic, esophageal, stomach, biliary tract, prostate, skin, lung, and brain cancer.

23. (canceled)

24. A method for synthesizing a compound of Formula I according to claim 1, the method comprising:
reacting a cyclopamine core with propargyl bromide in the presence of sodium carbonate to yield an alkyne-functionalized cyclopamine core,
reacting the functionalized cyclopamine core with an alkyl azide in a 1,3-cycloaddition reaction to form a cyclopamine conjugate, and
reacting the cyclopamine conjugate with a saccharide-azide in the presence of Cu(OAc)₂ and sodium ascorbate to form the compound of Formula I. saccharide

25. (canceled)

26. A compound comprising a salt, tautomer, dimer, solvate, complex or hydrate of a compound of Formula I according to claim 1.

27-32. (canceled)

33. A compound comprising a salt, tautomer, dimer, solvate, complex or hydrate of a compound of Formula II according to claim 8.

34-43. (canceled)

44. A method of treating cancer, the method comprising:
a) selecting a compound according to claim 1,
b) extracting cancer cells from a host,
c) evaluating the effectiveness of said compound in reducing the proliferation of the cancer cells against said cancer cells from the host,
d) repeating steps a through c until an effective compound is determined, and
e) treating said host with an effective amount of said effective compound.
